(19) United States
(12) Patent Application Publication
(10) Pub. No.: US 2026/0341405 A1
(43) Pub. Date: Oct. 29, 2026

(54) PREDICTING GENE EXPRESSION ALTERATIONS CAUSED BY PERTURBATIONS OF TRANSCRIPTION FACTORS

(71) Applicant: The Regents of the University of California, Oakland, CA (US)

(72) Inventors: Hongxu DING, Santa Cruz, CA (US); Joshua STUART, Santa Cruz, CA (US); Lucas SENINGE, Santa Cruz, CA (US)

(73) Assignee: The Regents of the University of California, Oakland, CA (US)

(21) Appl. No.: 18/997,402

(22) PCT Filed: Jul. 26, 2023
(86) PCT No.: PCT/US2023/028701

§ 371 Date: Jan. 21, 2025

Related U.S. Application Data
(60) Provisional application No. 63/392,315, filed on Jul. 26, 2022.

(51) Int. Cl. G16B 25/10; G06F 30/27; G16B 5/00; G16B 40/30

(52) U.S. Cl. CPC G16B 25/10; G06F 30/27; G16B 5/00; G16B 40/30

(57) ABSTRACT

Disclosed herein is an “in silico perturbation” Variational Autoencoder (VAE)-based deep learning framework. Such a framework can predict alterations in gene expression profiles caused by perturbations of transcription factor (TF) expression profiles. Perturbation strategies can be used to change a cell from one state to another state, e.g., one cell type to another cell type.

### CROSS-REFERENCES TO RELATED APPLICATIONS

The present application claims priority from and is a PCT application of U.S. Provisional Application No. 63/392,315, entitled “Predicting Gene Expression Alterations Caused By Perturbations Of Transcription Factors” filed Jul. 26, 2022, the entire contents of which are herein incorporated by reference for all purposes.

### BACKGROUND

Cells can exist in different states. Some states correspond to different types of cells, such as epithelial cells, immune cells, and neural cells. States can also capture different aspects of a cell, such as its stage in the cell cycle, its proliferation status, and its degree of maturation or differentiation. Tissues can be composed of different cell types and states, such as fibroblasts and muscle cells, diseased cells (e.g., tumor cells) and healthy cells, and cells in different diseased states. The different cell states can be reflected in expression profiles that quantify the amount of transcription from thousands of genes in a cell, which can be measured, for example, using RNA sequencing.

For biological and clinical applications, it may be desirable to change a cell from one state to another. For example, one may want stem cells to differentiate into a particular type of cell to reconstitute part of a tissue. In another example, one may want to change or eliminate a diseased cell. For example, a neoplastic or tumor cell may be converted to either a normal counterpart or an apoptotic cell that will undergo cell death. Experimental biologists can often modify the expression levels of a set of transcription factors (TFs) that control the expression of many other target genes using genetic manipulations, such as genetic knock-downs to lower the expression levels of TFs (e.g., via CRISPR or RNA-interference) or overexpression to increase the expression levels of TFs (e.g., via activating an inducible promoter). However, it is not known how to find the ideal expression levels of a combination of TFs to produce desired cell state changes.

### BRIEF SUMMARY

The disclosure provides methods, apparatuses, and systems of one or more computers that can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one example, a method may include performing one or more of the following operations on a computer system. The method include receiving an initial expression profile corresponding to an initial cell state, the initial expression profile may include initial expression levels for a set of transcription factors. The method also includes receiving a target expression profile corresponding to a target cell state. The method also includes loading, into memory, a decoder of a trained variational autoencoder (VAE), where the initial expression levels for the set of transcription factors correspond to a latent space of the trained VAE, and where the decoder generates a predicted expression profile using initial expression levels of the set of transcription factors. The method also includes adjusting one or more of the initial expression levels to obtain adjusted expression levels for the set of transcription factors. The method also includes using the decoder to generate an adjusted predicted expression profile from the adjusted expression levels, where the adjusted predicted expression profile corresponds to a current cell state. The method also includes determining a difference metric between the current cell state and the target cell state by comparing the adjusted predicted expression profile to the target expression profile.

In another example, a method may include performing one or more of the following operations on a computer system. The method may include loading an encoder of a variational autoencoder (VAE) into memory, where the encoder generates a latent space using an initial expression profile that may include expression levels for a set of transcription factors. The method also includes loading a decoder of the VAE into the memory, where the decoder generates a predicted expression profile using the latent space. The method also includes training the encoder and the decoder using a training set of expression profiles defining training cell states, where the training uses (1) a loss term including a difference between initial expression profiles and predicted expression profiles for the training set of expression profiles, and (2) a penalty term that constrains the latent space to match the expression levels for the set of transcription factors of the training set.

Other embodiments of this aspect include corresponding computer systems, apparatus, and computer programs recorded on one or more computer storage devices, each configured to perform the actions of any of the methods described herein.

A better understanding of the nature and advantages of aspects of the present disclosure may be gained with reference to the following detailed description and the accompanying drawings.

### BRIEF DESCRIPTION OF THE DRAWINGS

FIG. 1 shows an overview of variational autoencoder (VAE) architecture with interpretable latent space.

FIG. 2 shows an example architecture of VAE with constraint of latent space to be transcription factors.

FIG. 3 shows a an example workflow overview of in silico TF perturbation analysis.

FIG. 4 is a flowchart illustrating a method of training a VAE.

FIG. 5 shows the leveraging of TF regulatory information for in silico screening to identify downstream TFs on a regulatory network (e.g., TRRUST network).

FIG. 6 shows prediction of gene expression level alterations caused by perturbation on TF expression levels.

FIG. 7 is a flowchart illustrating a perturbation strategy for identifying adjustments of latent space expression values for transcription factors (TFs) to change an initial cell state to a target cell state.

FIG. 8 shows prioritization of effective perturbation strategies.

FIGS. 9 and 10 show results for perturbation of NOTCH-associated TFs.

FIGS. 11A-11B show quality control plots indicating that the method is working.

FIG. 12A shows lineage transition TFs highlighted on the TF volcano plot. FIG. 12B shows the original (circle) and perturbed (cross) cells projected on the PCA (principal component analysis) plot. FIG. 12C shows perturbation effects were projected on the assessment plot.

FIG. 13A shows Gata1-Spi1 module targets on a volcano plot. FIGS. 13B-13C show PCA and assessment plots showing perturbation effects.

FIG. 14 shows a heatmap of GMP genes in GMP cells and MEP cells.

FIG. 15 shows a heatmap of MEP genes in GMP cells and MEP cells.

FIG. 16A shows Top 5 highly expressed TFs in MEP and GMP. FIG. 16B is a PCA plot for the top-ranked Gata1-Cebpa combination.

FIG. 17 shows TF combinations ranked by perturbation strengths.

FIG. 18 is a flowchart illustrating a process for identifying an expression profile of a set of transcription factors that would correspond to a cell state that matches the target cell state.

FIG. 19 illustrates a measurement system according to an embodiment of the present disclosure.

FIG. 20 shows a block diagram of an example computer system usable with systems and methods according to embodiments of the present disclosure.

### DETAILED DESCRIPTION

Deep learning architectures such as variational autoencoders (VAEs) can be used to analyze transcriptomics data. However, the latent space of these VAEs offers little to no interpretability. To provide biological insights, some embodiments use a sparse variational autoencoder architecture. The VAE can include fully connected layers or other types of layers that are not fully connected, e.g., layers that include gene modules, as described herein. The decoder wiring of the VAE can mirror gene modules-a set of genes that function in concert to influence cell state changes—providing direct interpretability to the latent variables. For example, a gene membership mask M can be used to select a subset of trainable weights in the linear decoder layer that are determined by a given set of gene modules, provided either by the user or selected by the algorithm. Each latent variable can be viewed as a specific gene, and thus may be referred to as a gene module variable (GMV).

The genes in the latent space are selected to be transcription factors that can be adjusted to change an output expression profile for the decoder of the VAE. If the decoder can be trained to provide realistic expression profiles, then changes in the transcription factors can provide accurate prediction for the expression profiles of cells states that can be achieved with the change to the transcription factors. The decoder can be trained as part of a VAE where the latent space is constrained to be the transcription factors in the input expression level, while having the input expression levels match the output expression levels.

Accordingly, disclosed herein is an “in silico perturbation” Variational Autoencoder (VAE)-based deep learning framework. Such a framework is designed to predict alterations in gene expression profiles caused by perturbations of transcription factor (TF) expression profiles. Such a framework can be used to search for TF perturbation strategies that can yield desired gene expression patterns, e.g., changing a cell from an unhealthy state to a healthy state. The optimal values for the TFs can be determined using an optimization technique that has achieved a convergence criterion, e.g., the least difference between output expression profile and a target expression profile corresponding to a target cell state.

Once an optimal perturbation is determined for the transcription factors (TF) to achieve a target cell state, experiments can be performed to actually adjust the transcription factors to confirm the target state is achieved.

### I. CELLULAR MANIPULATION

The ability to change one type of cell to another type or to change one cell state to another cell state can be tremendously useful for many biological and therapeutic applications. For example, being able to precisely change a cancerous cell to a healthy cell or an apoptotic cell can provide therapeutic options that were not previously available. Methods of genetic manipulation have provided ways to alter cell states by, for example, knock-ins or knock-outs of different genes to change gene expression levels. However, it remains difficult to know the ideal expression levels of one or more genes that can produce a desired cell state.

### A. Transcription Factors

A transcription factor (TF) gene refers to a gene that encodes a certain transcription factor protein, which regulates the expression of other genes. Examples of TF genes can include genes annotated as TF genes in the Gene Ontology database, such as genes annotated with GO: 0003700, GO: 0003677, or GO: 0030528. As described in detail further herein, a target gene can be any gene whose expression level can be controlled by a TF. An expression profile is a numeric array, in which each element in the array represents the expression value of a specific feature.

As used herein, a TF expression profile is a numeric array where each element of the array represents the expression value of a specific TF gene. An output expression profile is a numeric array where each element of the array represents the expression value of a gene based on the TF expression as determined using a decoder of a VAE. As examples, the number of TFs can be in the range of a couple hundred of genes but can be narrowed down to 50-100 that have detectable and varying signals in a dataset. As a broader list of examples, the number of TFs can be at least 50, 60, 70, 80, 90, 100, 200, 500, 1,000, or 2,000. The number of target genes represented in the output numeric array can be in the tens of thousands (e.g., there are approximately 20,000-25,000 protein-coding genes in the genome) but may be restricted to those with detectable and varying expression, e.g., in the 500-2000 range of genes. Example numbers of target genes are provided elsewhere in this disclosure, and include at least 5, 10, 15, 20, 25, 30, 50, 100, 150, 200, 250, 300, 500, 1,000, 2,000, 5,000, 10,000, 15,000, 20,000, or 25,000 target genes.

A TF perturbation strategy can specify candidate TFs to be perturbed, as well as their perturbation direction (activation/repression) and perturbation magnitude. A space of perturbation strategies of the transcription factors can be searched to determine a TF expression profile that is consistent with a target expression profile for a cell. By using training techniques described herein, the decoder of the VAE can provide realistic output expression profiles where the TF expression profiles in the latent space are consistent with the actual, larger expression profile in the cells' states. A perturbation TF expression profile can correspond to the change from an initial TF expression profile to a target TF expression profile (corresponding to a target expression profile for the cell). Thus, the perturbation TF expression profile can specify the changes needed to the TF factors such that a cell changes from initial cell state to the target cell state.

### B. Cell States

As described herein, a cell state can refer to any characteristic or feature of the cell. A cell state can refer to a general feature of the cell, such as the cell type or the health or disease status of the cell. A cell state can also refer to one aspect of the cell, such as the shape of the cell, the metabolic state of the cell, or the modification of a particular protein within the cell (i.e., the phosphorylation status of a particular protein within the cell). The cells can be from any organism, such as humans, mammals, animals, plants, and fungi.

In some embodiments, an initial expression profile comprising initial expression levels for a set of transcription factors corresponds to an initial cell state. The target expression profile can correspond to a target cell state. As described herein, the initial cell state and the target cell state are different. For example, the initial cell state can be a diseased state (e.g., a cancerous cell state; also referred to as a cancer cell), and the target cell state can be an apoptotic state (i.e., a dead or dying cell). As another example, the initial cell state can be a diseased state (e.g., a cancerous cell state; also referred to as a cancer cell), and the target cell state can be a healthy state. As further examples, the initial cell state can correspond to a first type of cells, and the target cell state can correspond to a second type of cells, where the first type and the second type are different. Examples of various types of cells include, but are not limited to, epithelial cells, immune cells, neural cells, muscle cells, stem cells, bone cells, blood cells, and fat cells.

Once a subset of transcription factors, whose perturbation TF expression profile causes a change from one cell state to a target cell state, is found using the decoder of the VAE described herein, various gene editing tools can be further utilized to specifically target these transcription factors in vitro and/or in vivo. Examples of gene editing tools include, but are not limited to, gene editing by clustered regularly interspaced short palindromic repeats (CRISPR), Zinc Finger Nucleases (ZFNs) (see, Urnov et al., Nat. Rev. Genet., 11:636-646 (2010)), and transcription activator-like effector nucleases (TALENs) (see, Joung and Sander, Nat. Rev. Mol. Cell Biol., 1:49-55 (2013)). In particular, expression levels of specific transcription factors found using VAE described herein can be done using the gene editing tool CRISPR. The term “CRISPR” refers to any one of the naturally occurring Clustered Regularly Interspaced Short Palindromic Repeat systems or loci, or a derivative thereof. CRISPR loci can be found in the genomes of many bacteria and archaea. There are four types of CRISPR systems (e.g., Type I, Type II, Type III, and Type U).

A CRISPR locus can comprise polynucleotide sequences encoding for CRISPR Associated Genes (Cas) genes. Cas genes can be involved in the biogenesis and/or the interference stages of crRNA function. Cas genes can be named according to the organism from which they are derived. For example, Cas genes in Staphylococcus epidermidis can be referred to as Csm-type, Cas genes in Streptococcus thermophilus can be referred to as Csn-type, and Cas genes in Pyrococcus furiosus can be referred to as Cmr-type.

The term CRISPR nuclease refers to a polypeptide of, or derived from, a nuclease encoded in any one of the four types of CRISPR loci: Type I, Type II, Type III, and Type U, wherein the natural sequence of the polypeptide exhibits RNA-guided nuclease activity. In some embodiments, a CRISPR nuclease can be catalytically active or inactive. Catalytically inactive CRISPR nucleases do not exhibit nuclease or nickase activity when in complex with an RNA-guide and bound to a nucleic acid target containing a target domain and, in certain embodiments, a PAM sequence. The catalytically inactive CRISPR nuclease can be catalytically inactive due to one or more mutations of the CRISPR nuclease polypeptide sequence, or due to forming a complex with a guide RNA that is sufficient to provide RNA-guided targeting, but insufficient to support catalytic activity (i.e., nuclease or nicking activity). For example, the CRISPR nuclease can be a wild-type CRISPR nuclease (e.g., a Cas9 or Cpf1 nuclease) in complex with a dead guide sequence. For example, Cpf1 is a Class II CRISPR-Cas system and is described in Zetsche et al., Cell, 163:759-771 (2015). Dead guide sequences and their use are further described in, e.g., WO 2016/094872, which is hereby incorporated by reference for all purposes, including dead guide sequences, complexes between CRISPR nucleases and dead guide sequences, and methods and compositions for making and using such dead guide sequences and complexes containing them.

In certain embodiments, a CRISPR nuclease meets one or both of the following criteria: it has at least 20, 30, 40, 50, 55, 60, 65, 70, 75, 80, 81, 82, 83, 84, 85, 86, 87, 88, 89, 90, 91, 92, 93, 94, 95, 96, 97, 98, 99, or 100% sequence similarity with, or it differs by no more than 1, 2, 3, 4, 5, 6, 7, 8, 9, 10, 11, 12, 13, 14, 15, 16, 17, 18, 19, 20, 25, 30, 35, 40, 35, 50, 55, 60, 65, 70, 75, 80, 85, 90, 95, 100, 150, 200, 250, 300, 350 or 400, amino acid residues from, the amino acid sequence of a reference sequences, e.g., a naturally occurring CRISPR nuclease. Additional CRISPR nucleases include, without limitation, one or more CRISPR nucleases described in WO 2016/154579.

In certain embodiments, a CRISPR nuclease contains (i.e., is covalently or non-covalently linked to) one or more additional polypeptides or nucleic acids. For example, the CRISPR nuclease can be fused at an amino or carboxy-terminus to one or more transcriptional activation domain polypeptides, one or more DNA-binding polypeptides, one or more affinity tags (e.g., in complex with one or more affinity tag ligands, such as affinity tag ligand-transcriptional activation domain fusion protein(s)), nuclear localization sequences, or a combination thereof.

Accordingly, example perturbation strategies for changing a cell from one state to another include CRISPR activation (e.g., to activate the transcription factors directly and reprogram the cells, as in Weltner, J., Balboa, D., Katayama, S. et al. Human pluripotent reprogramming with CRISPR activators. Nat Commun 9, 2643 (2018). doi.org/10.1038/s41467-018-05067-x). Another example is viral delivery of cDNA, e.g., delivery of transcription factors to reprogram cells, such as fibroblasts to pluripotent stem cells, using retroviruses as delivery of cDNA plasmid containing the transcription factors (Takahashi K, Yamanaka S. Induction of pluripotent stem cells from mouse embryonic and adult fibroblast cultures by defined factors. Cell. 2006 Aug. 25; 126 (4): 663-76. doi: 10.1016/j.cell.2006.07.024. Epub 2006 Aug. 10. PMID: 16904174). Another example uses small molecules (e.g., drugs and chemical compounds) as may be used to differentiate stem cells into neurons with Noggin (a peptide) and SB431542 (a drug) to inhibit SMAD pathway. (Chambers, S., Fasano, C., Papapetrou, E. et al. Highly efficient neural conversion of human ES and iPS cells by dual inhibition of SMAD Nat Biotechnol signaling. 27, 275-280 (2009). https://doi.org/10.1038/nbt.1529). In some implementations, the molecules might not target transcription factors per se, but instead target the signaling pathway governed by transcription factors.

### II. DEEP LEARNING FRAMEWORK (VAE)

The disclosure provides a decoder of a variational autoencoder (VAE), where the decoder is trained to generate a gene expression profile based on the expression levels of various TFs. The VAE can be trained to provide an output expression level that matches the input expression level. A VAE architecture where a latent space can be constrained to correspond to certain genes is first described. Then, the selection of the latent gene space to be transcription factors (particularly those that may be adjusted) is described, where the training constrains the latent space to match the TF factors in the initial expression profile. Such a constraint provides a decoder that can accurately predict the expression profile of a cell based on input expression values of a set of TF factors.

### A. Variational Autoencoder (VAE)

Recent advances in single-cell RNA sequencing (scRNA-Seq) technologies have enabled the characterization of cellular states at an unprecedented scale and resolution. Among the many widely-used frameworks for analyzing complex transcriptomic patterns in single cells, artificial neural networks (ANNs) such as autoencoders (AEs) have emerged as powerful tools. AEs are neural networks that transform an input dataset into a decoded representation while minimizing the information loss. The diversity in their architectural design makes AEs suitable to tackle various important challenges of scRNA-Seq analysis, such as dimensionality reduction, clustering, and data denoising.

More recently, deep generative models such as variational autoencoders (VAEs) have proven to be extremely useful for the probabilistic modeling of single-cell transcriptomes, such as scVI and scGen. While standard AEs learn to reconstruct an input dataset, deep generative architectures explicitly model and learn the true data distribution, which allows a broader set of queries to be addressed. While deep generative models have shown impressive performance for their dedicated modeling tasks, they often lack interpretability thus cannot offer a biologically meaningful latent representation of transcriptomes. For example, latent perturbation vectors extracted with scGen cannot be directly related to gene module variations.

Integration of prior knowledge about gene modules to aid interpretability has already been successfully applied to transcriptomics data. DCell is a deep neural network integrating the hierarchical information about the molecular subsystems involved in cellular processes to guide supervised learning tasks, such as predicting growth in yeast. Such a model yields an informative biological interpretation of predictions by investigating the activation of the different subsystems embedded in the model's architecture. However, this model only works in a supervised learning setting where the goal is to predict a phenotypic outcome. On the other hand, f-scLVM is a Bayesian hierarchical model with explicit prior biological knowledge specification to infer the activity of latent factors as a priori characterized gene modules. While this approach enables the modeling of single-cell transcriptomes in an interpretable manner, the computational cost of the inference algorithm, as well as the absence of inference for out-of-sample data, make the development of more efficient approaches highly desirable.

In some embodiments, a VAE can offer an interpretable latent space to represent various biological information, e.g., the status of biological pathways or the activity of transcriptional regulators. Various embodiments can (1) encode data over an interpretable latent space and (2) infer gene module activities for out-of-sample data.

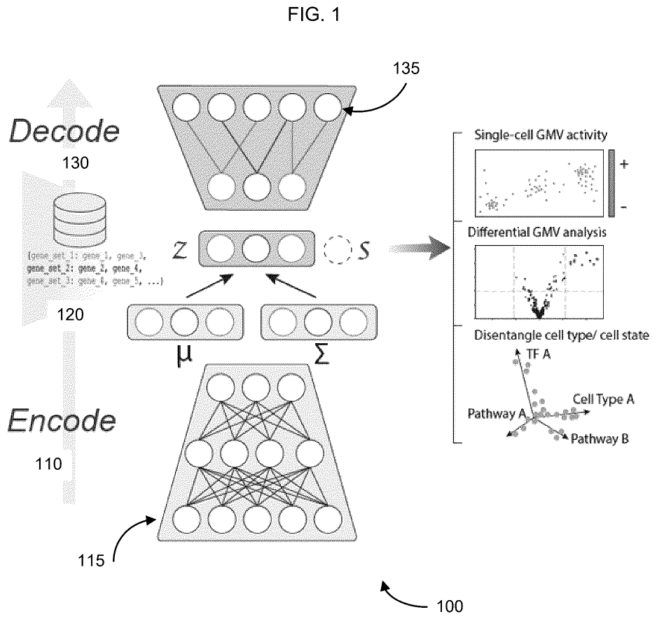

FIG. 1 shows an example variational autoencoder (VAE) 100 with interpretable latent space. VAE 100 includes an encoder 110, a latent space 120, and a decoder 130. An input expression profile 115 is provided to encoder 110, and an output expression profile 135 is provided by decoder 130. Latent space 120 is smaller than the size of the input and output expression profiles. VAE 100 is composed of a deep non-linear encoder (μ, Σ), representing a set of Gaussian distribution parameters that each generate a latent variable in the hidden layer z, and a masked linear decoder linking z to the output profile 135. VAE 100 can use an interpretable lower-dimensional latent space z that approximates a set of user-supplied gene modules (GMV). In embodiments described herein, the set of variables in z can be represented by a set of transcription factors (TFs). A fully connected decoder can be used or the TFs can also be treated as a GMV layer in which the z variables that mirror TF expression are only connected to their known target genes. Additionally, the VAE can integrate batch information as another variable s to condition its generative process on batch labels.

In a readily interpretable VAE, the decoder (generative part) connections of the neural network can be guided by gene module membership as recorded in gene annotation databases (e.g., Gene Ontology, PANTHER, MolSigDB, or Reactome). In many standard VAE implementations, the information bottleneck of the encoder-decoder architecture often represents latent variables modeled as a multivariate normal distribution. Despite providing highly informative representations of the input data, VAE latent variables are in general hard to interpret.

VAE 100 can implement a sparse architecture that explicitly reflects knowledge about gene regulation. In the service of biological pathways, genes work together in gene modules, regulated by common transcription factors that often produce correlated expression. Thus, if a given scRNA-Seq dataset X reflects the patterns of known gene modules, then it is possible for a VAE to learn a compact representation of the data by incorporating those modules as latent variables Z. VAE 100 can use multiple layers to approximate the latent variable distribution and produce a low dimensional, nonlinear representation of the original feature data.

The first and last layers (e.g., input expression profile 115 and an output expression profile 135) directly connect to the input or predicted features and so can be fashioned to depict intuitive groupings. Standard VAEs use a fully connected layer for both the encoding first layer and the decoding final layer. Instead, VAE 100 can use a gene membership mask M to select a subset of trainable weights in the decoder layer that are determined by a given set of gene modules (see Section V for further details). The mask can be applied to the weights that connect to the predicted output features to yield an interpretation of the latent variable layer where each latent variable is viewed as a specific gene module, henceforth referred to as a gene module variable (GMV).

The generative part of VAE 100 (i.e., decoder 130) can maintain a link from a GMV to an output gene only if this gene is annotated to be a member of this specific gene module. The two main advantages of this design are (1) the latent variables are directly interpretable as the activity of biological modules and (2) the flexibility in the gene module specification allows it to generalize to different biological abstractions (such as pathways, gene regulatory networks (GRNs), or even cell types) and can be taken from any of several curated databases of gene sets (such as MSigDB, Reactome pathways, inferred GRNs). Additionally, VAE incorporates information about covariates such as technical replicates in its latent space. This can be used to alleviate batch effects, as it has been demonstrated in previous deep generative models for single-cell data.

FIG. 1 also shows single-cell GMV activity, differential GMV activity, and a result of disentangling cell type/state. The data shows the ability to separate out GMV activity and different cell types/states.

B. VAE with Constrained Latent Space

Example VAEs described herein can contain an interpretable latent space. In some embodiments, the latent space comprises a set of transcription factors. For example, each value in the latent space can correspond to a different transcription factor. As examples, the set of transcription factors can comprise at least 10, 15, 20, 25, 30, or more transcription factors (e.g., up to 300 or more). As another example, the transcription factors can modify various numbers of target genes, e.g., at least 5, 10, 15, 20, 25, 30, 50, 100, 150, 200, 250, 300, 500, 1,000, 2,000, 5,000, 10,000, 15,000, 20,000, or 25,000 target genes.

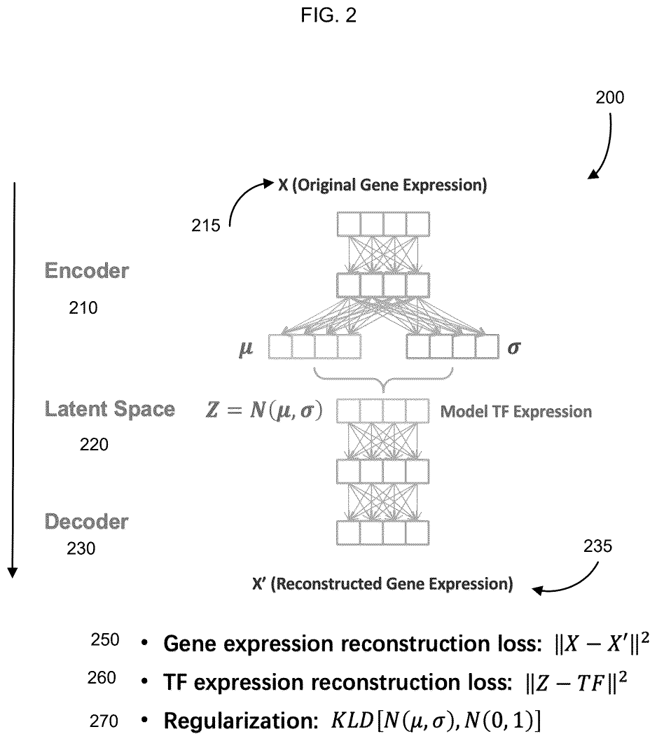

FIG. 2 shows an architecture of a VAE 200 with a constraint of the latent space to be transcription factors. VAE 200 includes an encoder 210, a latent space 220, and a decoder 230. An input gene expression profile 215 is provided to encoder 210, and an output expression profile 235 is provided by decoder 230. Latent space 220 is smaller than the size of the input and output expression profiles, as for VAE 100 (although not shown in FIG. 2). Thus, as shown in FIG. 2, the in silico perturbation deep learning framework includes three key components: the encoder neural network, the latent space, and the decoder neural network.

To train the model, an input gene expression profile 215 can be reduced to a latent space 220 of the TFs by an encoder 210. A decoder 230 can then generate the predicted gene expression profile 235 from TF-only expression levels. A trained model should predict a result that equals the input, so input gene expression profile 215 would be roughly equal to output expression profile 235 once VAE 200 is trained. The parameters of the encoder 210 and decoder 230 are optimized to ensure the matching of the input and output expression profiles. A loss function can include a difference between the two profiles, and parameters of encoder 210 and decoder 230 can be determined using an optimization technique for a set of training samples, i.e., different input expression profiles that are used. The loss function can include all of the errors (differences) for the training samples, and new parameters can iteratively be determined to minimize the loss function. In some embodiments, the decoder includes multiple fully connected layers of a neural network.

In the example show, a modified VAE loss function is used. The modification adds a TF expression reconstruction loss term 260 that constrains the latent space to match the observed expression levels of the TFs, e.g., in input gene expression profile 215.

As shown in FIG. 2, the gene expression reconstruction loss term 250 constrains the input (X) and output (X′) layers. Both X and X′ can represent the expression of all genes. Gene expression reconstruction loss term 250 quantifies the difference between an actual expression level and an expression level generated by the deep learning framework.

The latent space (Z) represents the expression of all TFs. A latent space, also known as a latent feature space or embedding space, can be described as an embedding of a set of items within a manifold, in which similar items are positioned closer to one another in the latent space than less similar items. A latent space can also be defined as a manifold that is transformed from the original space. The TF expression reconstruction loss term 260 constrains the latent space (Z). This TF expression reconstruction loss term 260 quantifies the difference between the generated and observed gene expression levels only among the TF genes.

Further, a regularization term 270 can be used. For example, a Kullback-Leibler Divergence (KLD) term can be used. A KLD term can be used to enforce a prior form (e.g., standard multivariate Gaussian with diagonal covariance) on the posterior distribution parametrized by the latent space Z. The KLD can quantify the difference between the prior and variational posterior distributions. Training a VAE

The disclosure also provides training a VAE using a training set of expression profiles defining training cell states. The encoder in the VAE can generate a latent space using an initial expression profile that comprises expression levels for a set of transcription factors. The decoder in the VAE can generate a predicted expression profile using the latent space. The training also uses (1) a loss term including a difference between initial expression profiles and predicted expression profiles for the training set of expression profiles, and (2) a penalty term that constrains the latent space to match the expression levels for the set of transcription factors of the training set. In some embodiments, once the initial expression profile and the predicted expression profile match, the loss term will approach zero, e.g., within a convergence criterion. In some embodiments, once the initial expression profile and the predicted expression profile match, the penalty term will be zero or none.

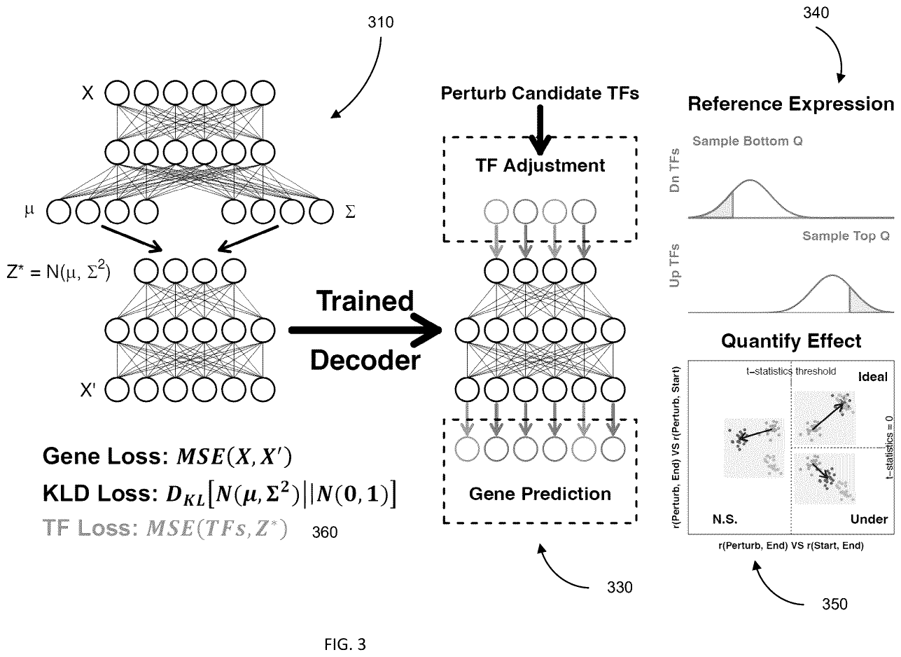

FIG. 3 shows an example workflow overview of in silico TF perturbation analysis. As shown in FIG. 3, a VAE 310 can model the generative process from TF expression to whole transcriptomes. X refers to the input expression profile and X′ to the output expression profile In some embodiments, to do so, we constrain the latent space to approximate TF expression by applying a TF loss term 360 during training, as described above for other figures. The trained decoder network is used to predict gene expression after TF perturbations, as shown in diagram 330.

Such predictions can start with adjusting the latent space Z*. Diagram 330 shows the TF adjustment being used by the decoder to predict a resulting expression profile.

Diagram 340 shows sampling of possible transcription factor expression values for the latent space. For TFs to be up-and down-regulated, corresponding latent values can be sampled from the top and bottom Q quantile of a reference expression profile. To infer the expression profiles after perturbation, a profile of possible expression values can be used. Sampling from the top Q (e.g., 1%, 2%, 3%, 4%, 5%, 10%, 15%, or 20%) and bottom Q (e.g., 1%, 2%, 3%, 4%, 5%, 10%, 15%, or 20%) is to impute biologically meaningful data. By selecting from a reference profile for a given transcription factor (e.g., where profile is obtained from a variety of cell types), one can ensure that the transcription factor can indeed be adjusted to that value, as opposed to selecting a biologically impossible value. The reference expression profile for a given gene (e.g., a transcription factor) across various cell types can be determined by measuring expression levels (e.g., as described herein) of one or more samples for each of the cell types. In some instances, a particular cell type can be used, e.g., if there is a strong biological prior.

Adjusted profiles can be subsequently flowed through the decoder network for final results. The relative strength among r (Perturb, End), r (Start, End) and r (Perturb, Start) Pearson correlations, quantified by t-statistics, can be used to assess if a perturbation yields the desired transcriptomic phenotype. Assessment plot 350 can be used to distinguish ideal, compromised (under) and non-significant (N.S.) groups. Start, End and Perturb represent the original, target and perturbed groups, respectively.

### C. Training Samples for VAE (Encoder and Decoder)

Training samples can include various expression profiles for various cell states. As described herein, a goal of the VAE is to output a same expression profile that is input to the VAE. The decoder will later be used to predict expression profiles corresponding to a cell state for an input of a certain set of transcription factors. Thus, to make the decoder accurate in predicting the expression profiles for various cell states, the set of training samples should correspond to various cells states.

For example, a certain portion of the training samples can correspond to expression profiles measured from healthy cell states. Another portion of the training samples can correspond to expression profiles measured from diseased cell states. Some implementations can train the VAE to be specific for a particular disease. For example, the diseased cells states can correspond to cancer. The expression profiles could be for a single tissue type, e.g., focusing on cancers of the liver. In this way, the predictions might be more accurate. However, the VAE would be of more narrow utility.

The training samples from which the training expression profiles are obtained can be selected based on the desired goal for the perturbation strategy. For example, the training samples can be selected based on the goal to change cells from stem cells into any number of specific tissue cells. Another example is to select cells for cancer, as described above. The cells of these training samples can be subjected to expression measurements, e.g., sequencing of RNA, hybridization capture onto arrays or beads, or in-situ florescence labelling in the cells. Any suitable type of gene expression quantification can be used.

### D. Loss Term

The training determines parameters of the VAE that reduce a loss function, the loss function can have various terms, as are described herein. At each iteration, new parameters can be determined based on the current loss (error) between the predicted and actual expression profiles. The new parameters can be determined using various techniques, e.g., gradient techniques, such as steepest descent (e.g., back propagation) or conjugate gradient, or Hessian techniques, such as Newton or quasi-Newton techniques. The parameters depend on the exact architecture and design of the VAE, e.g., multiplication coefficients and functional form for the activation functions (e.g., sigmoid functions, logistic functions, tanh or hyperbolic tangent functions, and ReLU (Rectified Linear Unit) functions).

One loss term includes a difference between initial expression profiles and predicted expression profiles for the training set of expression profiles, e.g., as shown in FIG. 2 as gene expression reconstruction loss term 250. This loss term helps to train the VAE such that the initial expression profile of the encoder eventually matches the predicted expression profile of the decoder. Gene expression reconstruction loss term 250 can be a log-likelihood term. Minimizing the reconstruction error can be a specific case of VAE where the generative process is Gaussian. Other VAEs can minimize other negative loglikelihoods.

In some embodiments, the VAE is trained using a VAE loss term that constrains a latent space of the VAE to match the initial expression levels. For example, TF expression reconstruction loss term 260 can constrain the latent space Z (e.g., latent space 220) to have values that are the same as the transcription factors. Each value in the latent space can be designated as corresponding to a particular TF. Thus, each for each input expression level, the array of values for the TFs can be generated, e.g., by using the value for TF 1 in the first value of the array and so on. This would be done for each input expression profile.

The TF expression reconstruction loss term (e.g., TF expression reconstruction loss term 260) can be summed over all of the training samples. For each input expression profile for a given iteration of the VAE, the expression reconstruction loss term can be determined. For a given input expression profile, the TF values can be extracted and used in the constraint term for that training sample; each training sample would have a different set of TF values in this reconstruction term. Once an aggregate (e.g., a sum) of the errors (losses) for each of the samples is determined, a set of new parameters for the VAE can be determined to reduce the total loss, which can include other terms.

Accordingly, the training can use a penalty term that constrains the latent space to match the expression levels for the set of transcription factors of the training set. The resulting decoder can be used to predict accurate output expressions based on the input TFs, as a result of this training.

### E. Training Method

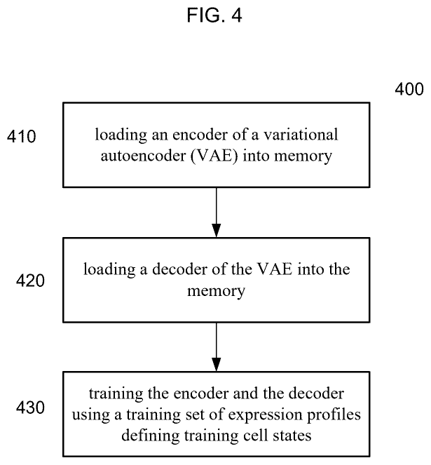

FIG. 4 is a flowchart illustrating a process 400 for training the VAE. As described above, the VAE can be trained for the purpose of obtaining a decoder that can predict an expression profile (e.g., of all genes in a panel that defines a cell state) using a smaller expression profile of transcription factors. The training can be performed on a variety of different types of computer systems, e.g., as described herein.

At block 410, an encoder of a variational autoencoder (VAE) is loaded into memory. The encoder can generate a latent space using an initial expression profile that comprises expression levels for a set of transcription factors. The initial expression profile can correspond to an expression profile for a set of genes that define a cell state. Such an initial expression profile includes more genes than just the set of transcription factors.

At block 420, a decoder of the VAE is loaded into the memory. The decoder can generate a predicted expression profile using the latent space. The predicted expression profile has more values that the latent space. The predicted expression profile can be of the same size as the initial expression profile. The encoder and decoder can include various layers, e.g., fully connected layers of a neural network. As another example, a sparsely connected set of connections reflecting gene modules, pathways, or transcription factor regulons, of a neural network can be used, such as GMV layers. As examples, the encoder can include three layers and the decoder can include 3 layers, but the encoder and decoder can used different numbers of layers. Other example numbers of layers for the encoder or decoder include 2, 4, 5, 6, 7, 8, 9, 10, or more. In an embodiment using GMV, the decoder can have only one GMV layer.

In an example embodiment, a VAE can include three encoder layers, three decoder layers, and one latent layer. For the encoder and the decoder, the number of nodes per layer can be the number of genes in the dataset or just the input encoder layer and output decoder layer can have the number of genes and intermediate layers can have a number between the number of genes and the number of transcription factors. The number of genes can be determined based on the dataset. Example datasets can have any value between 5,000 to 25,000 genes. Example numbers of nodes in the network can be at least 5,000, 10,000, 20,000, 30,000, 40,000, 50,000, 75,000, 100,000 or more nodes. As recited herein, example numbers of transcription factors are at least 100, 200, 500, or 1,000.

At block 430, the encoder and the decoder are trained using a training set of expression profiles defining training cell states. The training set can correspond to various cell states. For example, a certain portion of the training expression profiles can correspond to healthy cell states, and a certain portion of the training expression profiles can correspond to diseased cell states. In other examples, different portions can correspond to different types of cells, e.g., a stem cell and a heart cell. There can be many different portions for different types of cell states, as described herein.

The training can use (1) a loss term including a difference between initial expression profiles and predicted expression profiles for the training set of expression profiles, and (2) a penalty term that constrains the latent space to match the expression levels for the set of transcription factors of the training set. For example, the loss term can correspond to gene expression reconstruction loss term 250. As another example, the penalty term can correspond to TF expression reconstruction loss term 260.

The training can be performed using various techniques, such as steepest descent (such as back propagation), gradient techniques, such as conjugate gradient, or Hessian techniques, such as Newton or quasi-Newton techniques. The training updates parameters of the models in an attempt to reduce the loss term. The parameters depend on the exact architecture and design of the VAE, e.g., multiplication coefficients and parameters for the activation functions (e.g., sigmoid functions, logistic functions, tanh or hyperbolic tangent functions, and ReLU (Rectified Linear Unit) functions).

The results of the training generate a latent space Z that can be used to predict gene expression profiles in silico. In some aspects, the deep learning framework learns the regulatory logic of TF-to-target interactions. As a result, the deep learning framework that has been trained as described above can make predictions of the gene expression profile that results from perturbation of TFs. A perturbation can be alteration of the expression of a single or multiple TFs. Due to the architecture and parameter settings resulting from training, changes to TFs will propagate to changes in the target gene expression profile.

### III. USE OF DECODER

As explained above, the trained VAE can output a same expression profile as is input to the VAE. And since the latent space corresponds to a set of transcription factors (TFs), the decoder can predict an expression profile for a set of genes (e.g., a larger set that can include the transcription factors), where such a predicted expression profile effectively predicts a cell state.

Given that the decoder can predict a cell state (i.e., predicted expression profile) for a given set of expression values for a set of TFs, a computer can change the TF values in an effort to obtain a desired expression profile (i.e., target state). Accordingly, the computer can adjust one or more TFs and generate a predicted gene expression profile, which can be compared to a gene expression profile of a target cell state.

### A. Selecting Latent Space

Real-world biological experiments are usually designed to investigate a specific master regulatory TF module. Downstream TFs, which are required for an effective prediction for an output expression profile, are in general not provided. To identify such downstream TFs, we referred to the TRRUST TF regulatory target database (Han, Heonjong, et al. “TRRUST v2: an expanded reference database of human and mouse transcriptional regulatory interactions.” Nucleic acids research 46.D1 (2018): D380-D386). Various techniques can be used to determine the TFs to be adjusted and thus be in the latent space.

To perform the in silico perturbation analysis, embodiments can use a portion of a majority of the functional transcription factors. In biology, transcription factors are often organized as a regulatory graph. There can be a transcription factor A that regulates factors BCD, and B can regulate factors XYZ, which forms a regulatory network. A goal can be to perturb a single master regulator.

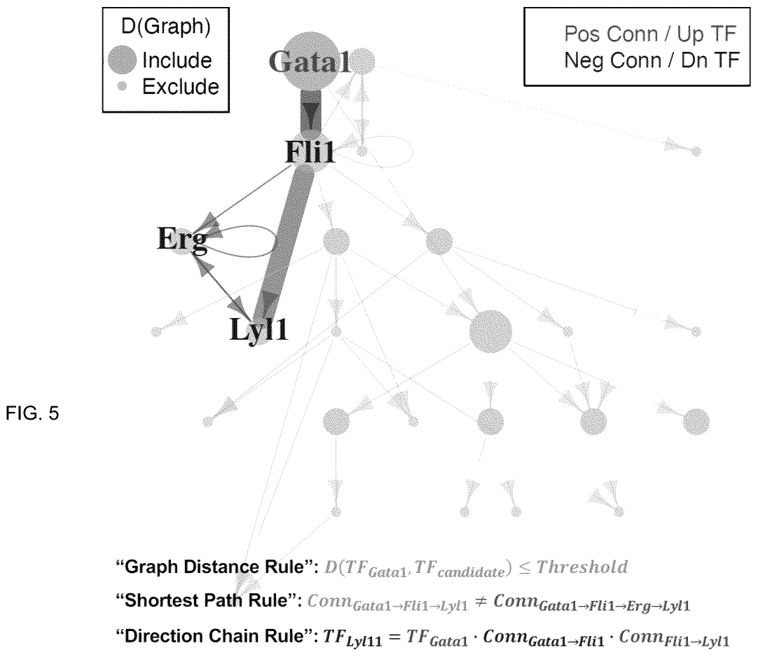

FIG. 5 shows the leveraging of TF regulatory information for in silico screening to identify downstream TFs on a regulatory network (e.g., TRRUST network). As shown in FIG. 5, target TFs are determined following three rules. First, only TFs within a certain graph distance radius are included (“Graph Distance Rule”). Second, only the shortest path to a target is used to calculate the regulatory direction (“Shortest Path Rule”). Third, the direction is calculated with a “Exclusive-NOR gate chain” (up-regulated TF/positive connection as 1, and vice versa) along the regulatory path (“Direction Chain Rule”).

In FIG. 5, GATA1 is a well-known master regulator. By following the biological regulator network, embodiment can identify the downstream transcription factors. The end of the regulatory network can provide the phenotype. The network in FIG. 5 can be used to search possible downstream targets, which can be used to determines the TFs for the latent space. Accordingly, as two examples, two ways to identify a candidate transcription factor for perturbing include searching the TF TRRUST network or searching the pathway gene sets.

Accordingly, embodiments can leverage a transcriptional regulatory network (e.g., TRRUST) to identify the full TF set downstream of a specific TF module. Embodiments can filter the network by only keeping TF-TF interactions with clear direction annotation. Embodiments can further remove duplicated edges and loops within the network. The cleaned-up network can then be used for determining target TFs and regulatory directions. As mentioned above, some examples can follow three rules, including “Graph Distance Rule”, “Shortest Path Rule” and “Direction Chain Rule”. In one implementation, the graph distance threshold is set as 6 for mouse hematopoiesis analyses. Even constrained by the “Shortest Path Rule”, multiple paths might be identified between two TFs. For such cases, embodiments can take the average regulatory directions calculated from the “Direction Chain Rule” as final direction assignments. The rationale being that, if both positive and negative regulations exist, their regulatory effect should cancel out and the direction with more known paths should dominate. We took TFs with positive and negative “average directions” as up and down-regulated targets.

### B. Adjusting Latent Space

The TF values in the latent space can be adjusted, thereby providing a predicted expression profile. As the TF values are adjusted, a new expression profile would be predicted. The TF factors can repress or active genes.

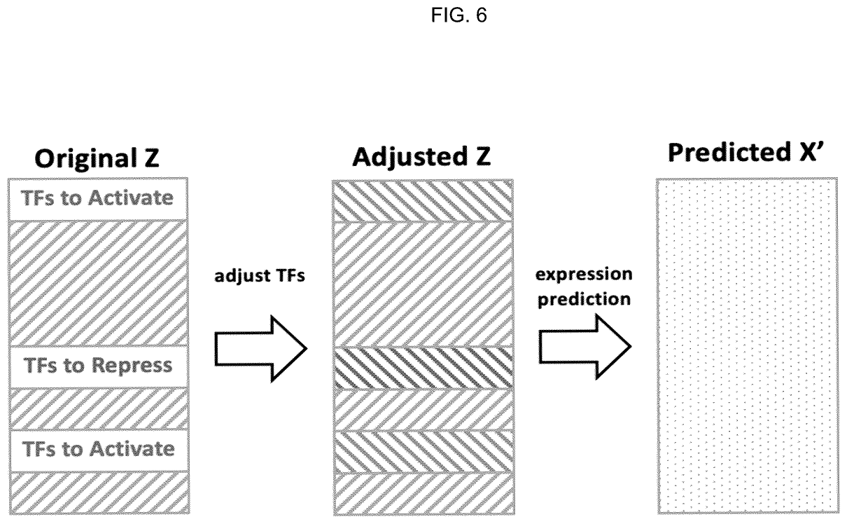

FIG. 6 shows a prediction of gene expression level alterations caused by perturbation on TF expression levels. For a given TF perturbation strategy, performing the in silico perturbation analysis is shown. In the latent space Z derived from the training of the VAE, the expression levels associated with selected candidate TFs (Original Z) can be adjusted, thereby creating adjusted TF expression levels (Adjusted Z). Based on the adjusted Z (corresponding to a new set of expression values for the TFs), an expression profile can be predicted. Accordingly, the predicted gene expression profile (Predicted X′) can be obtained by propagating the expression levels of the adjusted TFs through the decoder of the trained VAE. Although the predicted X′ is shown as the same size as the latent space, the number of expression values for predicted X′ is typically larger.

Thus, the latent space can comprise a number of TFs. Adjusting the latent space can include selecting a subset of TFs from the initial set of TFs. The latent space can include all of the initial set of TFs or only the subset. Regardless, only a subset of the TFs might be changed in some examples. As shown, only about one third of the TFs are adjusted in FIG. 6.

### C. Perturbation Strategies

As described above, the initial expression levels for the set of transcription factors correspond to a latent space of the trained VAE. The latent space can be adjusted, with a new output expression profile predicted for the adjusted expression values of the TFs. The adjustment can continue to as part of a search process to identify a latent space that corresponds to a target expression profile, e.g., of a target cell state. Accordingly, a computer can perform a search by iteratively adjusting a subset of the transcription factors to obtain adjusted expression levels for the set of transcription factors that brings the predicted expression profile closest to the target expression profile.

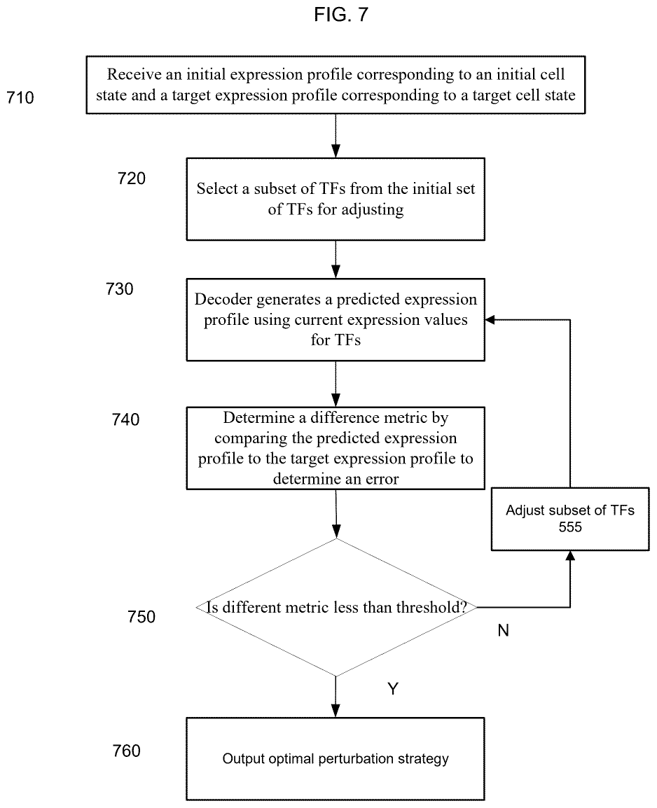

FIG. 7 is a flowchart illustrating a search process 700 for identifying adjustments of latent space of expression values for transcription factors (TFs) to change an initial cell state to a target cell state. The adjustments can be made so that a target expression profile of the target cell state is achieved. Process 700 may be done partially or entirely by a computer system.

At block 710, an initial expression profile is received corresponding to an initial cell state. The initial expression profile includes a subset of transcription factors (TFs).

At block 720, a subset of TFs from the initial set of TFs are selected for adjusting. There can be more TFs in the latent space, so that the subset of TFs adjusted may only be a portion of the total number of TFs in the latent space. The ones selected for adjusting may be TFs where techniques are known to be available for adjusting the gene. Example techniques (e.g., CRISPR) for adjusting the TFs are described in other sections herein. As another example, TF modulators (such as drugs) may be available.

At block 730, the decoder generates a predicted expression profile using current expression levels of the set of transcription factors. For the first iteration, the current expression levels are the initial expression levels. Thus, the decoder generates an adjusted predicted expression profile from the adjusted expression levels, in which the adjusted predicted expression profile corresponds to a current cell state.

At block 740, the predicted expression profile is compared to the target expression profile to determine a difference metric. The difference metric can reflect an error between the predicted expression profile and the target expression profile. For example, if the predicted expression profile was (1, 4, 2) and the target expression profile was (1, 2, 5), then the difference metric might be 5, which is a sum of the absolute value of the differences. The error can take many forms, such as a simple difference of each value, a ratio, a weighted difference (e.g., each expression value could have a different weight), or functions thereof.

At block 750, it is determined whether the difference metric is less than a threshold. The comparison can show whether the iterative process has converged to be sufficiently close to target cell state. The threshold value might vary based on what the initial cell state and the target cell state are. For example, if the target cell state is a healthy cell state then the threshold might be less than if the target cell state is a different type of cell.

At block 755, if the difference is more than the metric, the search process proceeds to adjust the subset of TFs. The amount of adjustment for each of the subset of TFs can be determined based on the specific differences for which expression values. The adjustments can be selected to make the largest reduction in the difference metric. The skilled person will appreciate the type of optimization techniques that can be used, e.g., gradient techniques or a brute force search can map out different values for the space of TF that are selected for changing. The search can use variable step sizes in gradient or other directions, e.g., by selecting two or more step sizes and performing a parabolic fit to approximate a local minimum in a selected direction (e.g., a vector corresponding to the changes in the TFs that are selected for changing). The search process would then proceed back to block 730 to generate a new predicted expression profile for the current latent space of expression values for the TFs.

At block 760, if the difference metric is less than the threshold, the optimal perturbation strategy is provided. The optimal perturbation strategy can provide a sufficient low difference between the initial values for the TFs and the final values for the set of TFs. Thus, the adjusted expression profile generated by the decoder can eventually match the target expression profile, thereby providing a strategy for how to change the actual genes corresponding to the TFs to change the cell from an initial state to the target state.

The in silico perturbation deep learning framework can be used to prioritize effective perturbation strategies. Specifically, candidate perturbation strategies can be ranked by quantifying the similarity between their yielded gene expression profile predictions and a pre-specified target gene expression profile. Alternatively, the best strategy (e.g., highest similarity score or smallest difference to target expression profile) can be selected from a specified list of different sets (or subsets) of TFs. In both scenarios, iterative optimization can determine optimal adjustments for the subset of TFs that are being adjusted.

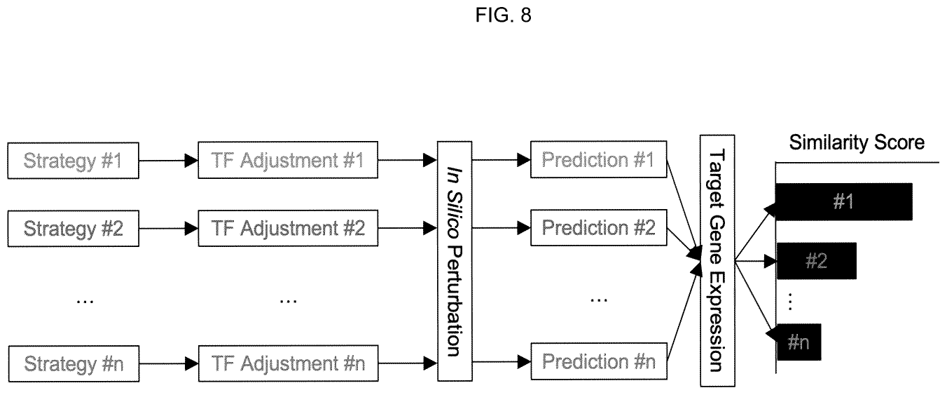

FIG. 8 shows a prioritization of effective perturbation strategies. Various candidate TF perturbation strategies can be tried (n as shown). Following steps shown in FIG. 7, based on the original TF expression profile (Original Z), an adjusted TF expression profile (Adjusted Z) can be created for each perturbation strategy, then the trained decoder of the VAE can be used to generate a predicted target gene expression profile (Predicted X′). The similarity score between yielded gene expression predictions and the target gene expression profile can be quantified to prioritize the effective perturbation strategies.

A subset of TFs can be different numbers of TFs. A subset of TFs can also be different types of TFs. For example, all of the TFs within the same genetic locus can be used. In another example, a subset of TFs can contain various TFs from different genetic loci. In some embodiments, depending on the different cell states to be altered, specific TFs can be selected that particularly target expression levels of the genes involved in the cell state. For example, if the initial cell state is a diseased state and the target cell state is an apoptotic state, TFs that change the expression levels of apoptotic proteins can be selected. Further, if the diseased state of the cell is known or speculated to be caused by one or more proteins, the TFs of those one or more proteins can be chosen to be in the subset and the changes in the expression levels of these TFs can alter the expression levels of the one or more proteins involved in the diseased state of the cell. In some embodiments, adjusting the latent space by choosing subsets TFs iteratively can be performed automatically. In other embodiments, adjusting the latent space by choosing subsets TFs iteratively can be performed manually.

### D. Results

As a proof-of-concept, the in silico perturbation deep learning framework was applied to prioritize perturbations that can drive Day0 cells (cellular state #1) to Day2 cells (cellular state #2). Biologically, such a Day0-to-Day2 cellular state transformation is driven by the NOTCH signaling pathway.

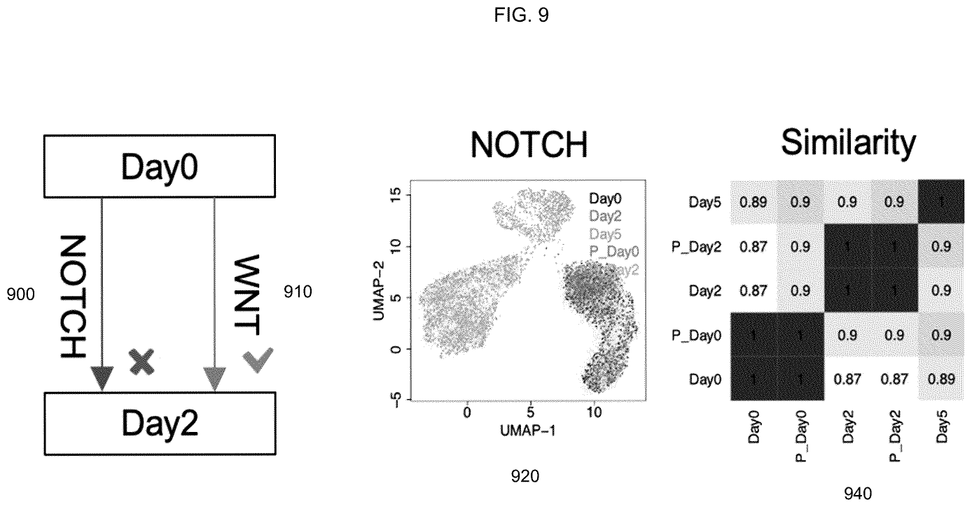

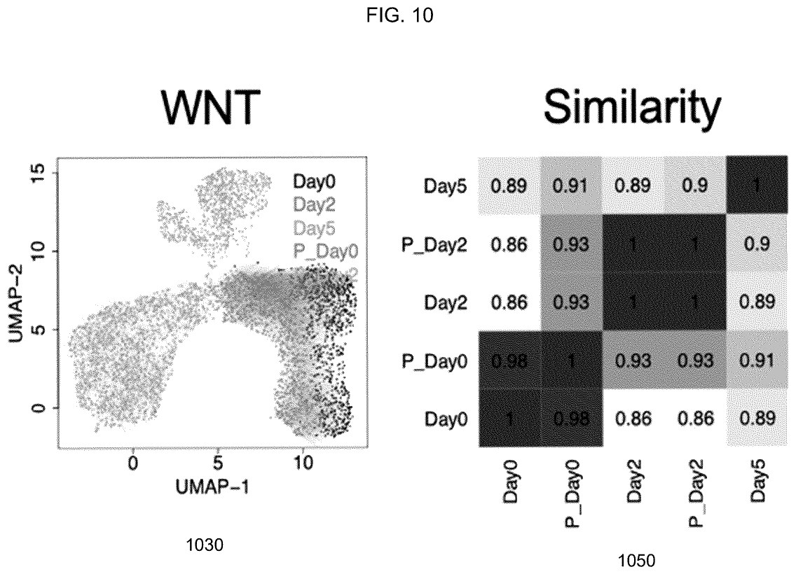

FIGS. 9-10 show results for perturbation of NOTCH-associated TFs and WNT-associated TFs. As shown in FIGS. 9-10, in silico perturbation on WNT-associated TFs pushed the Day0 cells towards the Day2 cellular state (P_Day0 in panel WNT) (910, 1030, 1040). As a negative control, another common but biologically irrelevant signaling pathway, NOTCH (900), was examined. As shown in FIG. 9, no obvious cellular state transformation can be observed (P_Day0 in panel NOTCH) when performing in silico perturbation on NOTCH-associated TFs (920, 940).

We performed further analysis using the TOME dataset (tome.gs.washington.edu). We obtained normalized TOME and mouse hematopoiesis expression profiles. For each cell population in TOME, we further filtered genes whose expression rates are less than 10%. We then took the union of the kept genes as the final gene list. Such a selection process aims at preserving genes that are uniquely expressed in small cell populations. TOME lineage transition-specific TF sets were collected from the Table S7 and S9 of (Qiu, Chengxiang, et al. “Systematic reconstruction of cellular trajectories across mouse embryogenesis.” Nature genetics 54.3 (2022): 328-341). We took the union of such TFs as the final TF list. The mouse hematopoiesis dataset as a whole was filtered under expression rates threshold 10%. For this dataset, we took the union of TOME and TRRUST TFs as the final TF list. We split the TOME dataset for training and benchmarking. The training and testing datasets were built by randomly sampling 200 and 100 cells per cell population, respectively. Considering the relatively small number of cells, the mouse hematopoiesis dataset as a whole was used for training and benchmarking.

### 1. TOME Analysis—Recovery of Transcriptome

We benchmarked using the TOME mouse embryogenesis single cell dataset (Qiu, Chengxiang, et al. “Systematic reconstruction of cellular trajectories across mouse embryogenesis.” Nature genetics 54.3 (2022): 328-341), which contains 468 cell populations and 113 lineages. We first evaluated the trained Manatee model and confirmed that the whole transcriptome could be recovered from TF expression.

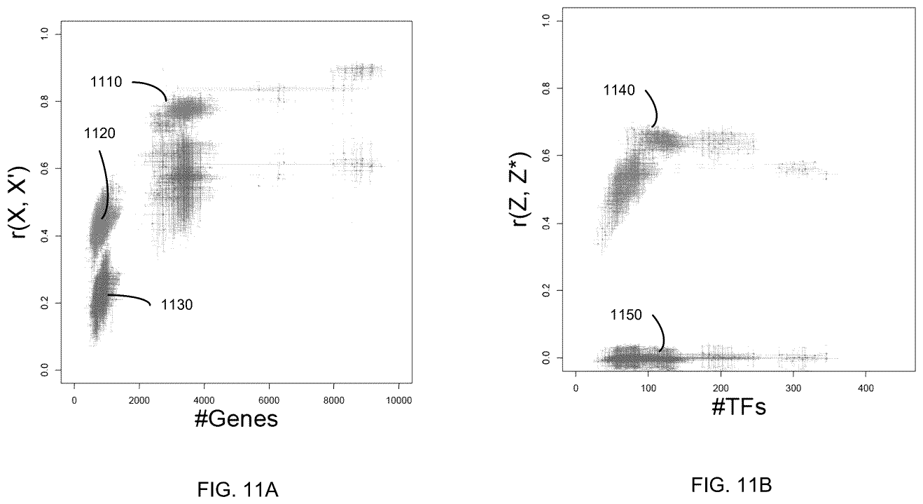

FIGS. 11A-11B show quality control plots indicating that the method is working. The plots show that the trained decoder can reconstruct gene expression. In FIGS. 11A-11B, r represents Pearson correlation. We use Pearson correlation to quantify the network since it is a variational encoder. The correlation relates to similarity between initial expression profile X and reconstructed expression profile X′. If the correlation is good, then that variation of the encoder network can reconstruct the expression matrix.

The predict data 1110 and 1140 reflects the training process. As one can see, the correlation between X and X′ increases from lower numbers of genes and TFs in the model as the numbers increase.

The generate data 1120 is the in silico perturbation of the latent space to generate the output expression profile. From the latent space Z, the decoder predicts X′. Thus, for a specific cell, from the transcription factor expression of the cell, we test whether you can reconstruct the training expression for the output expression profile.

The depend data 1130 and 1150 corresponds to a random sample of the latent space Z. By randomly sampling the expression of a TF across different cells, one will destroy the dependency between TFs. Then, inputting the decorrelated TF expression into the decoder, one can test the ability to reconstruct the gene expression. The performance should be compromised due to the random sampling, which is what is shown in FIGS. 11A-11B.

Overall, we observe that embodiments have the capability to encode diverse biological states all together, e.g., cell states and trajectories. We also observe that disrupting TF correlations by randomly shuffling TF profiles compromises transcriptome reconstruction as would be expected.

### 2. TOME Dataset-Confirmation of Trajectory

That TOME dataset records cell types and tlineage transitions in mouse embryogenesis. Embryogenesis is the really early developmental processes in organisms.

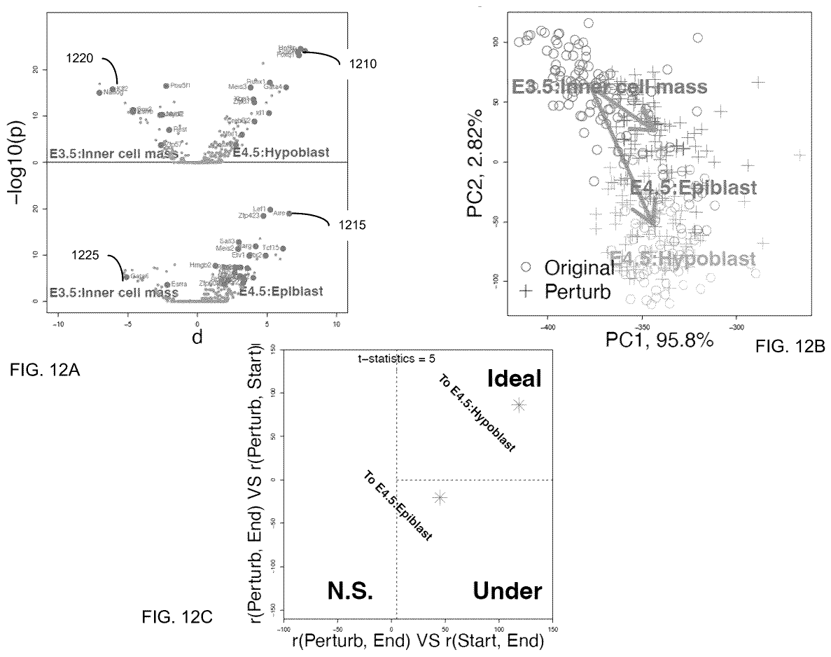

We tested whether in silico perturbation could recover lineage transitions. As an example, we analyzed the lineage bifurcation from inner cell mass to hypoblast and epiblast. We perturbed key TFs documented in TOME (FIG. 12A), and recovered both trajectories (FIGS. 12B and 12C). We further analyzed all 113 TOME lineages. As shown by this data, the trained VAE is capable of producing biologically meaningful transcriptomic predictions from in silico perturbed TF latent variables.

FIG. 12A shows lineage transition TFs highlighted on the TF volcano plot. Up-regulated (+) TFs 1210, 1215 and down-regulated (−) TFs 1220, 1225 in lineage targets (upper panel hypoblast and lower panel epiblast) are shown. P-values (vertical axis) were calculated using the two-sided U-test. D-values (horizontal axis) represent average TF expression differences. FIG. 12B shows the original (circle) and perturbed (cross) cells projected on the PCA (principal component analysis) plot. Colors represent cellular identities: inner cell mass (stem cell), epiblast, and hypoblast. Arrows indicate perturbation directions. FIGS. 12A-B show that if you perturb the annotated transcription factors, one will recover the lineage bifurcation, which provides a proof of concept of the technique. FIG. 12C shows perturbation effects were projected on the assessment plot.

### 3. Trrust

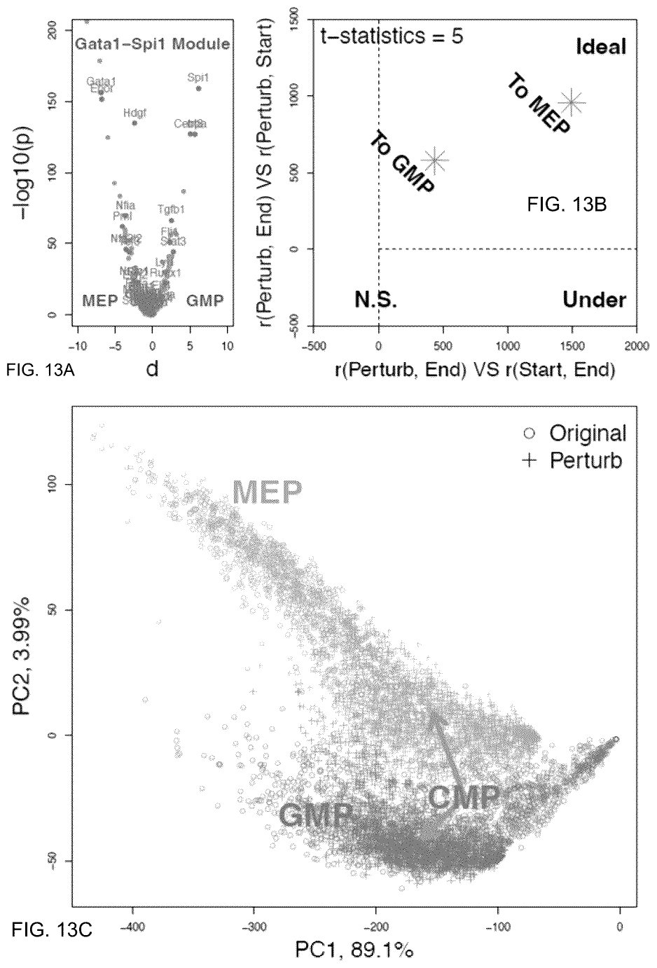

We benchmarked the TRRUST-powered analysis with the CMP to GMP VS MEP lineage bifurcation for hematopoiesis, which is driven by the Gata1-Spi1 module (Paul, Franziska, et al. “Transcriptional heterogeneity and lineage commitment in myeloid progenitors.” Cell 163.7 (2015): 1663-1677). Pu.1 (also referred to as Spi1) is an essential factor for reconstitution of the myeloid lineage (Graf and Enver, 2009) . . . while Gata1, Klf1, and Gfilb are essential for erythrocyte and megakaryocyte lineage commitment (Iwasaki et al., 2003; Orkin et al., 1998; Pevnyet al., 1991). Spi1 and Gata1 are experimentally validated master regulatory TFs for the lineage bifurcation. CMP is a progenitor cell. The progenitor cell can give rise to two different cell types, MEP and GMP. We trained and evaluated the mode, identified target TFs (FIG. 13A), and recovered both trajectories (FIGS. 13B and 13C).

FIG. 13A shows Gata1-Spi1 module targets on a volcano plot. Up and down-regulated targets between GMP and MEP were marked as red and blue, respectively. P-values and d-values were calculated described for FIG. 12A. FIGS. 13B-13C show PCA and assessment plots showing perturbation effects, as in FIGS. 12B-12C. As one can see after perturbation, the perturbed cells got closer to the target cells.

FIGS. 13A-13C shows a balance between GATA1 and Spi1: two mass regulators. As shown, the relative activity of these two mass regulators determines downstream targets of the GATA1 Spi1 module. The targets were perturbed based on whether the MEP path or the GMP path is desired. Given that GATA1 and Spi1 are experimentally validated (Franziska et al.), these data show the model works. Franziska et al. also describes an experimental decrease in the expression of Cebpa prevents the production of GMP, which is the evidence showing Cebpa to be the master regulatory TF, as shown in FIGS. 13A-13C.

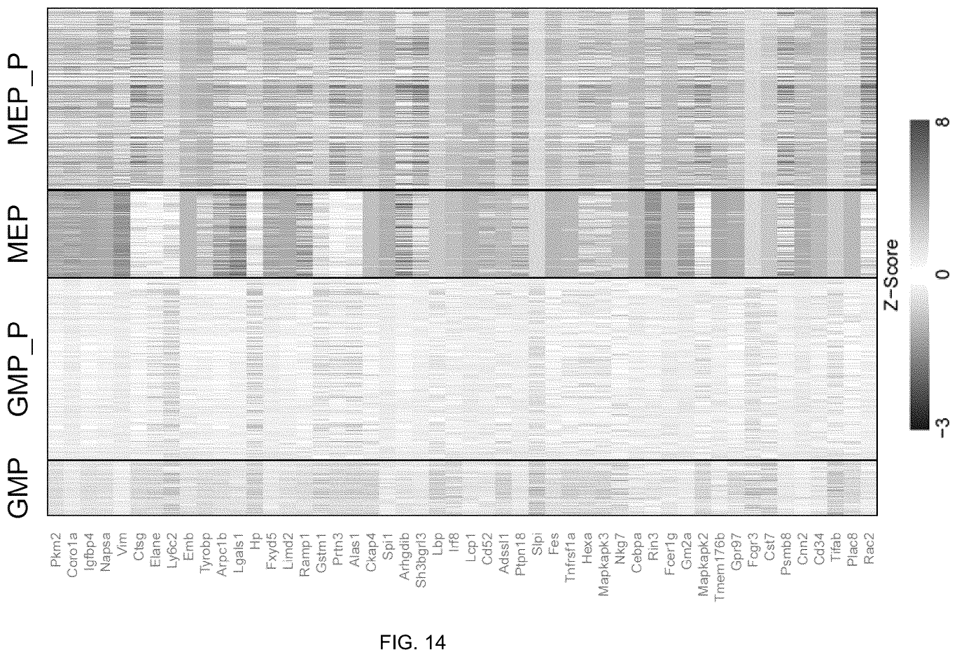

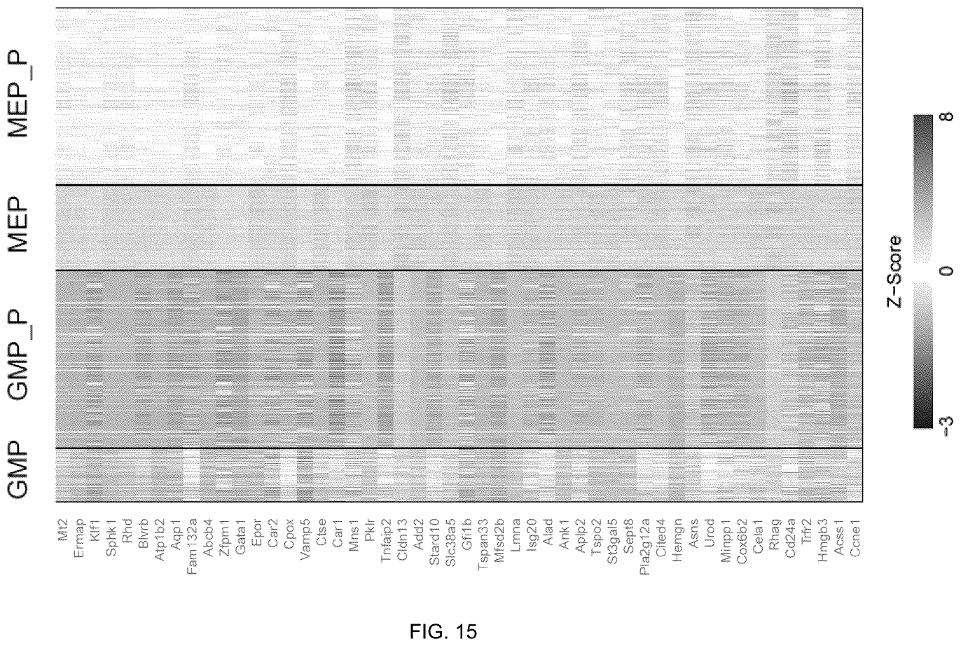

We further confirmed that in silico perturbation is biologically valid, by recovering cell type-specific marker genes (FIGS. 14-15), which shows each gene is correct. The MEP genes (downregulated in MEP) and the GMP genes (upregulated in GMP) were confirmed to have these expected values when the transcription factors were adjusted.

FIG. 14 shows a heatmap of GMP genes in GMP cells and MEP cells. As expected, the GMP genes are upregulated in the GMP cells and downregulated in the MEP cells. FIG. 15 shows a heatmap of MEP genes in GMP cells and MEP cells. As expected, the MEP genes are upregulated in the MEP cells and downregulated in the GMP cells.

FIGS. 14-15 show how the individual marker genes react, indicating whether the perturbation is working or not. To do this, we determined the top 50 highly expressed genes in each population: MEP population and GMP population. These are considered to be marker genes for that population, population specific genes, marker genes. Then after in silico perturbation, we tested the expression levels for these marker genes can be obtained in the desired cell type. FIGS. 14-15 show we can recover those marker genes after perturbation. The identity of the cell that we want to produce via this perturbation is preserved.

### 4. Selecting Best Perturbation Strategy

Above, we established a framework that can predict a perturbation effect. For example, for a perturbation, the model can provide what the gene expression is. The model can be used in a different way. For several possible perturbation strategies, the model can be used to determine which one provides the best matching gene expression profile for the target desired phenotype. Accordingly, we leveraged the model for the in silico screening of perturbations that could yield the target transcriptomic phenotype.

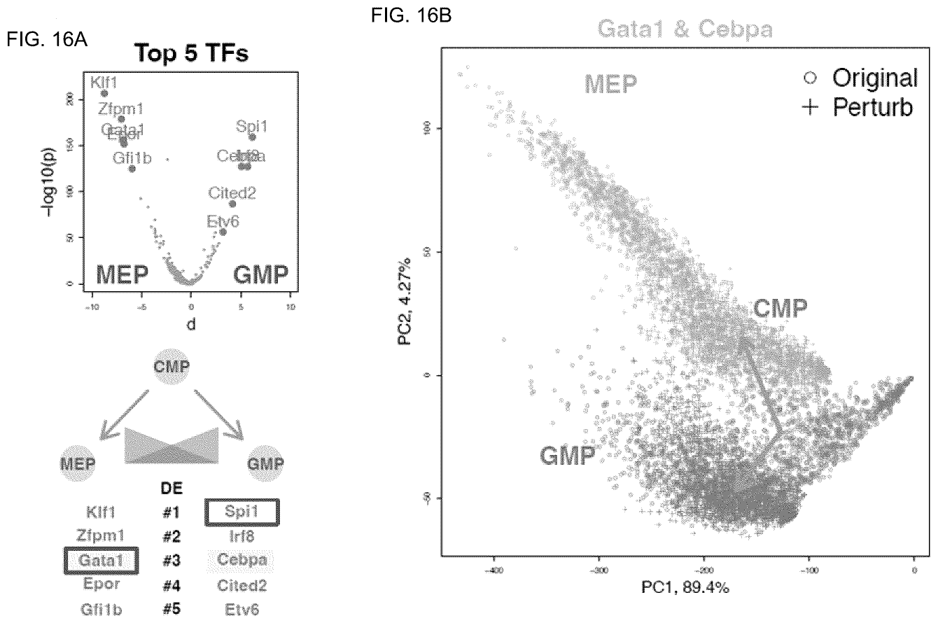

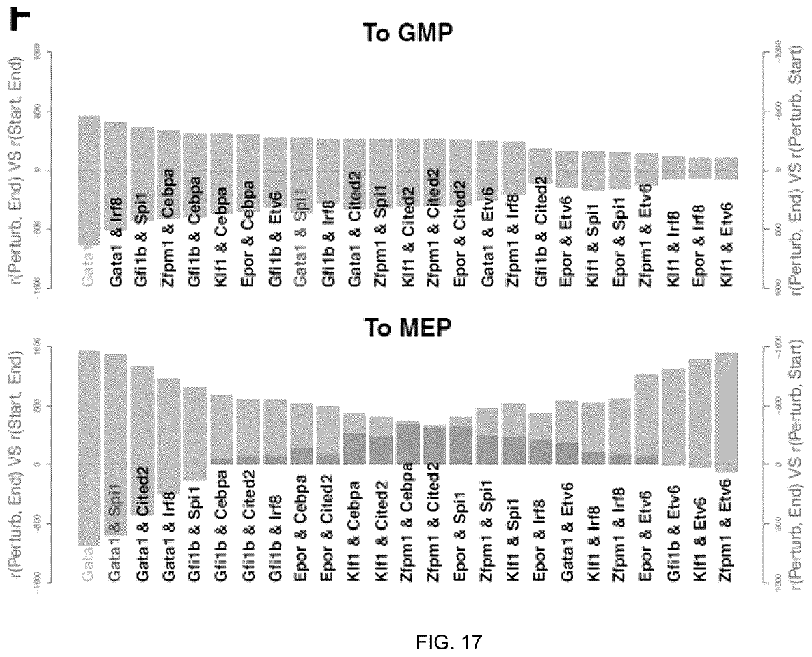

As a proof-of-concept, we screened alternative TF duos driving the above hematopoiesis process. Without losing generality, we screened all 25 combinations of the top 5 highly expressed TFs within MEP and GMP (FIG. 16A) and ranked them based on perturbation effectiveness (FIG. 17). We prioritized the Gata1-Cebpa combination, which could effectively guide lineage transitions (FIG. 16B) as well as recover marker gene expression. Noticeably, Cebpa has been experimentally validated as a master regulator controlling the identity of GMP (Paul, Franziska, et al.).

FIG. 16A shows Top 5 highly expressed TFs in MEP and GMP. FIG. 16B is a PCA plot for the top-ranked Gata1-Cebpa combination. FIG. 16B is a bifurcation plot showing at a global level, whole transcriptome level, the cell starts going to the right direction to MEP or GMP. FIG. 17 shows TF combinations ranked by perturbation strengths.

We screened all 81 possible combinations of four signaling pathways, and we prioritized the most feasible ones for producing stem cells. Since we know that GATA1 and Spi1 are the mass regulatory module for controlling the bifurcation. We determine whether there are alternative transcription factor pairs that can do that. We screened all 25 possible combinations of top five highly expressed transcription factors in each population. We found that Gata1 plus Cebpa is also a good combination. Franziska, et al showed that Cebpa is a mass regulator, which validates the present results.

### E. Method Using Decoder

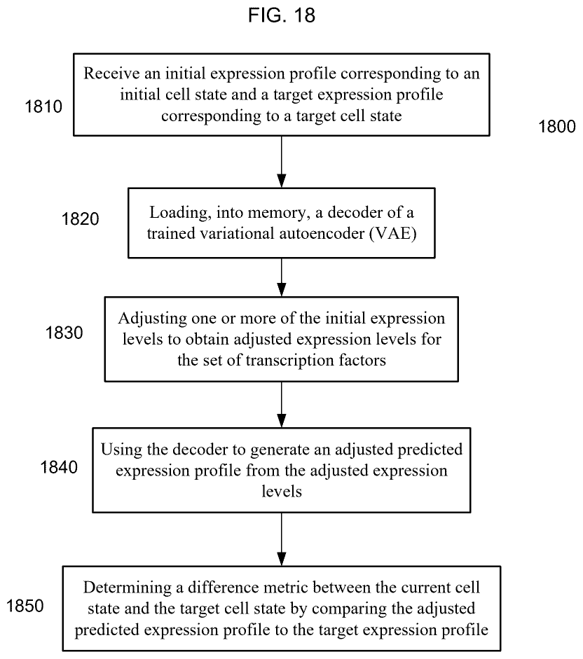

FIG. 18 is a flowchart illustrating a process 1800 for identifying an expression profile of a set of transcription factors that would correspond to a cell state that matches the target cell state.

At block 1810, an initial expression profile is received corresponding to an initial cell state and a target expression profile is also received corresponding to a target cell state. In some embodiments, the initial expression profile can comprise an initial set of transcription factors. In some embodiments, the target expression profile can comprise a subset of transcription factors that falls within the initial set of transcription factors. In some embodiments, a subset of the transcription factors is identified. These transcription factors can be adjustable for determining one or more candidate perturbation strategies to reach the target cell state.

In some embodiments, the initial cell state is a diseased state (e.g., a cancerous cell state) and the target cell state is an apoptotic state. In other embodiments, the initial cell state is a diseased state (e.g., a cancerous cell state) and the target cell state is a healthy state. Further, the initial cell state can correspond to a first type of cell and the target cell state can correspond to a second type of cell, and the first and second types are different. Examples of cell types include, but are not limited to, epithelial cells, immune cells, neural cells, muscle cells, stem cells, bone cells, blood cells, and fat cells.

At block 1820, a decoder of a trained variational autoencoder (VAE) is loaded into memory, in which the initial expression levels for the set of transcription factors correspond to a latent space of the trained VAE, and the decoder generates a predicted expression profile using initial expression levels of the set of transcription factors. In some embodiments, the predicted expression profile comprises a subset of transcription factors that falls within the set of transcription factors in the latent space.

At block 1830, the one or more of the initial expression levels are adjusted to obtain adjusted expression levels for the set of transcription factors. For the first iteration, the initial expression levels correspond to the initial cell state.

At block 1840, the decoder is used to generate an adjusted predicted expression profile from the adjusted expression levels, in which the adjusted predicted expression profile corresponds to a current cell state. In some embodiments, the process also includes performing a search by iteratively adjusting the subset of the transcription factors; and based on the search, recommending a perturbation strategy from a list of candidate perturbation strategies. In some embodiments, the recommended perturbation strategy provides a best match of the predicted expression profile to the target expression profile corresponding to the target cell state. The recommended perturbation strategy can be implemented on a physical cell having the initial cell state, e.g., using perturbation strategies described herein.

At block 1850, a difference metric between the current cell state and the target cell state is determined by comparing the adjusted predicted expression profile to the target expression profile. The difference metric can reflect an error between the adjusted predicted expression profile and the target expression profile. For example, if the adjusted predicted expression profile was (1, 4, 2) and the target expression profile was (1, 2, 5), then the difference metric might be 5, which is a sum of the absolute value of the differences. The error can take many forms, such as a simple difference of each value, a ratio, a weighted difference (e.g., each expression value could have a different weight), or functions thereof. In some embodiments, based on the metric difference, one can determine whether to further adjust the expression levels for the subset of transcription factors.

Process 1800 can identify perturbations that have aberrant phenotypes as side-effects, as the resulting cell after adjustment may have certain desired properties but also certain unwanted expression levels. For example, a drug can change a specified set of transcription factors. By perturbing these ten transcription factors, there can be a reversion of a cancer signature in a subject, but there can also be a different bad gene expression pattern, e.g., cell death.

In summary, the in silico perturbation can start with adjusting latent values corresponding to candidate TFs. In order to produce a legitimate latent space, TF values can be sampled from a reference matrix (distribution), which tracks expression patterns in actual biological settings. In this study, we used the full expression matrices as references. For TFs to be up and down-regulated, corresponding values can be sampled with replacement from the top and bottom Q quantile of the reference, e.g., where Q is 1% or other values mentioned herein. Adopting such an “extreme” Q can make sure only representative values are used. A rationale can be that 1) we observed that TF value distributions between original and target phenotypes are in general close to each other, and 2) a specific cell population only accounts for a very small proportion of the entire TOME collection. For TFs not marked as candidates, their original expression values can be kept the same.

### IV. EXAMPLE VAE DETAILS

A variational autoencoder (VAE) is trained to generate an expression profile based on the expression of TFs. In a VAE, the initial expression levels of one or more TFs corresponding to an initial cell state can be adjusted to generate a predicted expression profile, which can be compared to an expression profile of a target cell state.

To train the VAE, an input expression profile corresponding to an initial set of TFs can be reduced to a latent space of the TFs by an encoder. A decoder can then generate the predicted expression profile corresponding to a subset of the initial TFs. The parameters of the encoder and decoder are optimized using a modified VAE loss function. The modification adds a loss term that constrains the latent space to match the observed expression of the TFs. Example details of various embodiments of the VAE are provided below. Such example details may be optional.

### A. VAE Architecture

The VAE can maximize the likelihood of a single-cell dataset X under a generative process [7, 10] described as:

p(X|θ)=∫p(X|Z,θ)p(Z|θ)dZ, (1)
with θ being the learnable parameters of a neural network. As described above, a VAE can uses a set of latent variables Z that correspond to transcription factors. The latent variables can also or instead explicitly represent sets of genes (gene modules), such as pathways, gene regulatory networks or cell type marker sets. To enforce the VAE to interpret a dataset from the viewpoint of a set of gene modules, the decoder part can be made up of a single, masked, linear layer. Specifically, the connection of this layer, between latent node z^{(j)} and gene features, can be specified using a binary mask M in which Mi,j is true if gene i is a member of gene module j and false otherwise. Each latent variable z(j) can be referred to as a gene module variable (GMV) since each provides a view of the data constrained to the subset of genes for a distinct gene module j. During training, gradients associated with masked (false) weights can be “zeroed out” such that backpropagation only applies to weights originating from a user-supplied given gene set. Additionally, the weights of the decoder can be constrained to be non-negative (w≥0) to maintain interpretability as to the directionality of gene module activity.

Having explicitly specified the connections between genes and latent variables in the decoder of VAE (generative part), embodiments can incentivize that the latent space represents a biological module activity interpretation of the data. The GMVs can be modeled as a multivariate normal distribution, parametrized by our inference network with learnable parameters φ As such, the distribution of the Z latent variables can be expressed as:

q(Z|X,φ)= [symbol] (μ_{φ}(X),Σ_{φ}(X)) (2)

This choice of variational distribution is common and has proven to work well in previous single-cell studies [9, 10]. Following similar standard VAE implementations [7, 10], the objective to be maximized during training is the Evidence of Lower Bound (ELBO):

[symbol]

(X)= [symbol] _{q(z|X,φ)}[log p(X|Z,θ)]−KL(q(Z|X,φ)∥p(Z|θ)) (3)
where the expectation over the variational distribution can be approximated using Monte Carlo integration over a minibatch of data, and the Kullblack-Leibler divergence term has a closed-form solution as we set the prior to:

p(Z|θ)~ [symbol] (0,I) (4)

The reparameterization trick [7] is used when sampling VAE's variational distribution to allow standard backpropagation to be applied when training the model.

To retain information of genes that are not present in our pre-annotated biological networks, additional fully-connected nodes to the latent space can be used in the VAE. This has two effects: (1) it allows VAE to model the expression of unannotated genes, which could be crucial for a good reconstruction of the data during training, and (2) it can help capture additional variance of the data that is unexplained by the provided gene modules, considerably improving the training of the model. The number of additional fully-connected nodes can be determined based on a trade-off between model performances and the loss of information encoded by pre-annotated GMV nodes. As examples, 16 or fewer extra FC nodes can be used to preserve biological signal encoded by GMV nodes. In some instances, only fully-connected layers are used.

Additionally, the diagonal covariance prior used in the latent space modelling discourages GMVs from being correlated. Thus, the VAE may be forced to choose an arbitrary gene set among many equally informative but overlapping sets and could fail to reveal a key annotation. To address this issue, a dropout layer can be added to the latent space of the model. This has been shown to force the VAE to preserve redundancy between latent variables [34], which is applicable when the gene annotation database used to initialize latent space contains overlapping gene sets.

Finally, batch information or other categorical covariates can be encoded via extra nodes in the latent space, conditioning the generative process of VEGA on this additional covariate information.

Various embodiments described herein can be adapted from a VAE architecture (e.g., as described in Kingma, Diederik P., and Max Welling. “Auto-encoding variational bayes.” arXiv preprint arXiv: 1312.6114 (2013)) and be designed to model the generative process from TF profiles to corresponding transcriptomes. Both the encoder and decoder neural networks can comprise the same number of fully connected layers (e.g., three). Each layer can have the same number of nodes as the number of genes. The latent space can contains the same number of latent variables as the number of TFs. The following loss function can be optimized during training:

L=(1−α)×{(1−β×L_{R}+β×D_{KL}}+α×Lᵣ
where L_{R} and D_{KL} represent reconstruction loss and Kullback-Leibler Divergence against the N (0, 1) normal distribution respectively, as the two regular VAE loss terms. The additional Lᵣ term represents the TF reconstruction loss, which is the mean square error (MSE) between reparameterized latent variables (Z) and TF expression (TF):

Lᵣ≡MSE(Z,TF)

Coefficient α balances regular VAE loss terms L_{R} and D_{RL}, while coefficient β balances the regular VAE loss and the added TF reconstruction loss term Lᵣ.

As described above, example number of TFs can be at least 50, 60, 70, 80, 90, 100, 200, 500, 1,000, or 2,000. Example numbers of target genes are provided elsewhere in this disclosure, and include at least 5, 10, 15, 20, 25, 30, 50, 100, 150, 200, 250, 300, 500, 1,000, 2,000, 5,000, 10,000, 15,000, 20,000, or 25,000 target genes. Example numbers of nodes in the network can be at least 5,000, 10,000, 20,000, 30,000, 40,000, 50,000, 75,000, 100,000 or more nodes.

B. Measuring Differential GMVs Activity of the Latent Space with Bayes Factor (BF)

The difference in the activity of genes and/or pathways is often of interest when contrasting two different groups of cells. For a given GMV k, a pair of cells (xa, xb) and their respective group ID (sa, sb) (e.g., two different treatment conditions), our two mutually exclusive hypotheses are:

$${ℋ}_{0}^{k} := {𝔼}_{s} [ {z}_{a}^{k} ] > {𝔼}_{s} [ {z}_{b}^{k} ] vs . {ℋ}_{1}^{k} := {𝔼}_{s} [ {z}_{a}^{k} ] \leq {𝔼}_{s} [ {z}_{b}^{k} ] \qquad ( 5 )$$

This can intuitively be seen as testing whether a cell has a higher mean GMV activation than another, the expectation representing empirical frequency. We evaluate the most probable hypothesis by studying the log-Bayes factor K defined as:

$$K = {log}_{e} \frac{p ( {ℋ}_{0}^{k} \vert {x}_{a} , {x}_{b} )}{p ( {ℋ}_{1}^{k} \vert {x}_{a} , {x}_{b} )} \qquad ( 6 )$$

Here, the sign of K tells us which hypothesis is more likely, and the magnitude of K encodes a significance level. Having access to the conditional posterior distribution q(Z|X) over the GMVs activation (the encoding part of VAE), we can approximate each hypothesis' probability distribution as:

$$p ( {ℋ}_{0}^{k} \vert {x}_{a} , {x}_{b} ) \approx {\sum }_{s} p ( s ) {\int \int }_{sup . ( {z}_{a} ) , sup . ( {z}_{b} )} p ( {z}_{a}^{k} > {z}_{b}^{k} ) dq ( {z}_{a}^{k} \vert {x}_{a} ) dq ( {z}_{b}^{k} \vert {x}_{b} ) \qquad ( 7 )$$

where p(s) is the relative abundance of cells in group s, and the integrals are approximated with direct Monte Carlo sampling.

Assuming cells are independent, we can compute the average Bayes factor across many cell pairs randomly sampled from each group respectively. This helps us decide whether a GMV is activated at a higher frequency in one group or the other. Through the paper, we consider GMVs to be significantly differentially activated if the absolute value of K is greater than 3 (equivalent to an odds ratio of ≈20) [9, 20].

### VI. EXAMPLE SYSTEMS

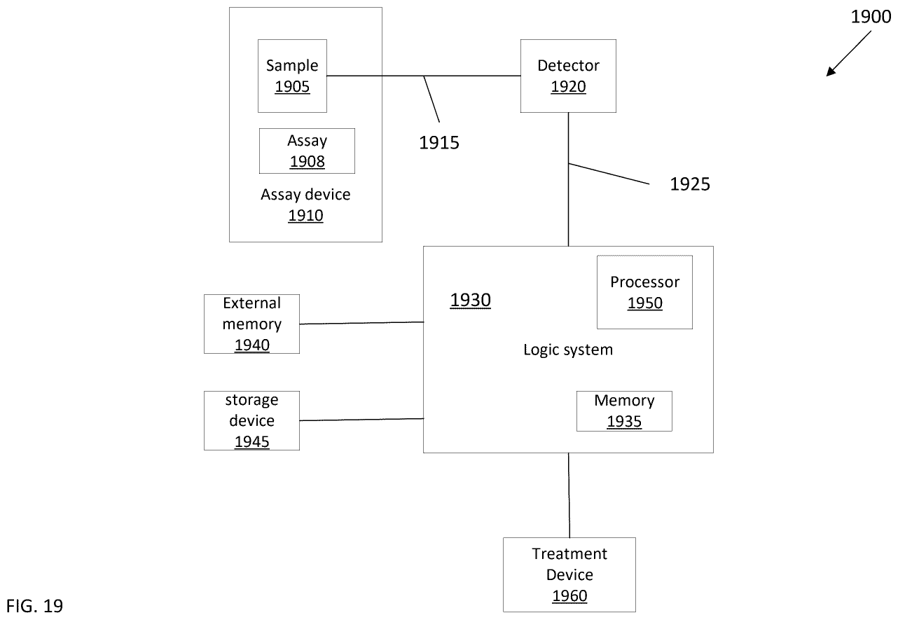

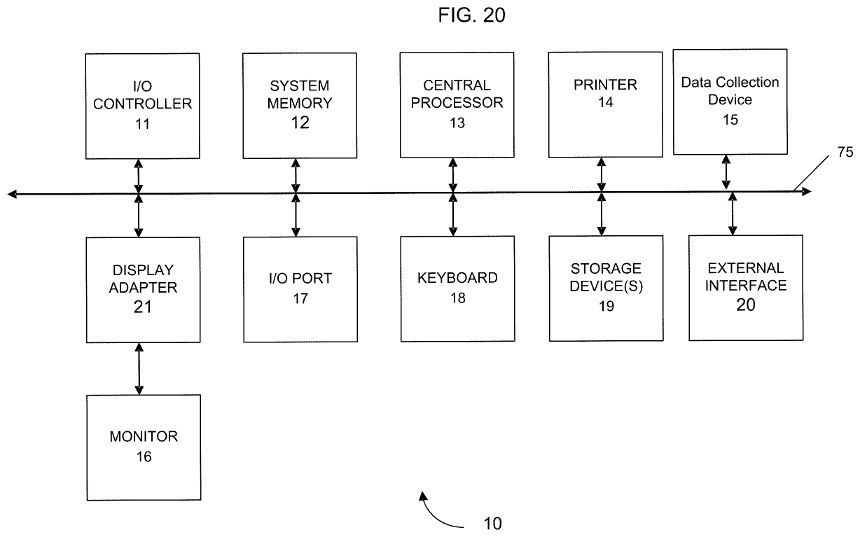

FIG. 19 illustrates a measurement system 1900 according to an embodiment of the present disclosure. The system as shown includes a sample 1905, such as nucleic acid molecules (e.g., DNA and/or RNA) within an assay device 1910, where an assay 1908 can be performed on sample 1905. For example, sample 1905 can be contacted with reagents of assay 1908 to provide a signal of a physical characteristic 1915 (e.g., expression levels of a set of genes in a sample). An example of an assay device can be a flow cell that includes probes and/or primers of an assay or a tube through which a droplet moves (with the droplet including the assay). Physical characteristic 1915 (e.g., a fluorescence intensity, a voltage, or a current), from the sample is detected by detector 1920. Detector 1920 can take a measurement at intervals (e.g., periodic intervals) to obtain data points that make up a data signal. In one embodiment, an analog-to-digital converter converts an analog signal from the detector into digital form at a plurality of times.

Assay device 1910 and detector 1920 can form an assay system, e.g., a sequencing system that performs sequencing according to embodiments described herein. A data signal 1925 is sent from detector 1920 to logic system 1930. As an example, data signal 1925 can be used to determine sequences and/or locations in a reference genome of nucleic acid molecules (e.g., DNA and/or RNA). Data signal 1925 can include various measurements made at a same time, e.g., different colors of fluorescent dyes or different electrical signals for different molecule of sample 1905, and thus data signal 1925 can correspond to multiple signals. Data signal 1925 may be stored in a local memory 1935, an external memory 1940, or a storage device 1945. The assay system can be comprised of multiple assay devices and detectors.

Logic system 1930 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1930 and the other components may be part of a stand-alone or network connected computer system, or they may be directly attached to or incorporated in a device (e.g., a sequencing device) that includes detector 1920 and/or assay device 1910. Logic system 1930 may also include software that executes in a processor 1950. Logic system 1930 may include a computer readable medium storing instructions for controlling measurement system 1900 to perform any of the methods described herein. For example, logic system 1930 can provide commands to a system that includes assay device 1910 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 1900 may also include a treatment device 1960, which can provide a treatment to the subject. Treatment device 1960 can determine a treatment and/or be used to perform a treatment. Examples of such treatment can include surgery, radiation therapy, chemotherapy, immunotherapy, targeted therapy, hormone therapy, and stem cell transplant. Logic system 1930 may be connected to treatment device 1960, e.g., to provide results of a method described herein. The treatment device can act upon a cell (e.g., treat a cell) using a perturbation strategy determined using embodiments described herein.

Any of the computer systems mentioned herein may utilize any suitable number of subsystems. Examples of such subsystems are shown in FIG. 10 in computer system 100. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 10 are interconnected via a system bus. Additional subsystems such as a printer 14, keyboard 18, storage device(s) 19, monitor 16 (e.g., a display screen, such as an LED), which is coupled to display adapter 21, and others are shown. Peripherals and input/output (I/O) devices, which couple to I/O controller 11, can be connected to the computer system by any number of means known in the art such as input/output (I/O) port 17 (e.g., USB, FireWire®). For example, I/O port 17 or external interface 20 (e.g., Ethernet, Wi-Fi, etc.) can be used to connect computer system 10 to a wide area network such as the Internet, a mouse input device, or a scanner. The interconnection via system bus allows the central processor 13 to communicate with each subsystem and to control the execution of a plurality of instructions from system memory 12 or the storage device(s) 19 (e.g., a fixed disk, such as a hard drive, or optical disk), as well as the exchange of information between subsystems. The system memory 12 and/or the storage device(s) 19 may embody a computer readable medium. Another subsystem is a data collection device 15, such as a camera, microphone, accelerometer, and the like. Any of the data mentioned herein can be output from one component to another component and can be output to the user.

A computer system can include a plurality of the same components or subsystems, e.g., connected together by external interface 20, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

### VII. REFERENCES

1. Latchman, David S. “Transcription factors: an overview.” The international journal of biochemistry & cell biology 29.12 (1997): 1305-1312.
2. Kingma, Diederik P., and Max Welling. “Auto-encoding variational bayes.” arXiv preprint arXiv: 1312.6114 (2013).
3. Lotfollahi, Mohammad, F. Alexander Wolf, and Fabian J. Theis. “scGen predicts single-cell perturbation responses.” Nature methods 16.8 (2019): 715-721.
4. Seninge, Lucas, et al. “VEGA is an interpretable generative model for inferring biological network activity in single-cell transcriptomics.” Nature communications 12.1 (2021): 1-9.
5. Kamimoto, Kenji, Christy M. Hoffmann, and Samantha A. Morris. “CellOracle: Dissecting cell identity via network inference and in silico gene perturbation.” bioRxiv (2020).
6. Hinton, G. E. “Reducing the dimensionality of data with neural networks.” Science 313, 504-507 (2006).
7. Baldi, P. “Autoencoders, unsupervised learning, and deep architectures.” Proc. Mach. Learn. Res. 27, 37_49 (2012).
8. Wang, D. & Gu, J. “VASC: dimension reduction and visualization of single-cell RNA-seq data by deep variational autoencoder.” Genomics, Proteomics Bioinformatics 16, 320-331 (2018).
9. Geddes, T. A. et al. “Autoencoder-based cluster ensembles for single-cell RNA-seq data analysis.” BMC Bioinformatics 20, 660 (2019).
10. Eraslan, G., Simon, L. M., Mircea, M., Mueller, N. S. & Theis, F. J. “Single-cell RNA-seq denoising using a deep count autoencoder.” Nat. Commun. 10, 390 (2019).
11. Way, G. P. & Greene, C. S. Extracting a biologically relevant latent space from cancer transcriptomes with variational autoencoders. Pac. Symp. Biocomput. 23, 80-91 (2018).
12. Lopez, R., Regier, J., Cole, M. B., Jordan, M. I. & Yosef, N. “Deep generative modeling for single-cell transcriptomics.” Nat. Methods 15, 1053-1058 (2018).
13. Lotfollahi, M., Wolf, A. F. & Theis, F. J. “scGen predicts single-cell perturbation responses.” Nat. Methods 16, 715-721 (2019).
14. Ma, J. et al. “Using deep learning to model the hierarchical structure and function of a cell.” Nat. Methods 15, 290-298 (2018).
15. Buettner, F., Pratanwanich, N., McCarthy, D. J., Marioni, J. C. & Stegle, O. “f-scLVM: scalable and versatile factor analysis for single-cell RNA-seq.” Genome Biol. 18, 212 (2017).
16. Subramanian, A. et al. “Gene set enrichment analysis: a knowledge-based approach for interpreting genome-wide expression profiles.” Proc. Natl Acad. Sci. USA 102, 15545-15550 (2005).
17. Jassal, B. et al. “The reactome pathway knowledgebase.” Nucleic Acids Res. 48, D498-D503 (2020).
18. Margolin, A. A. et al. “ARACNE: an algorithm for the reconstruction of gene regulatory networks in a mammalian cellular context.” BMC Bioinformatics 7, S7 (2006).

1. A method comprising performing, by a computer system:
receiving an initial expression profile corresponding to an initial cell state, the initial expression profile comprising initial expression levels for a set of transcription factors;
receiving a target expression profile corresponding to a target cell state;
loading, into memory, a decoder of a trained variational autoencoder (VAE), wherein the initial expression levels for the set of transcription factors correspond to a latent space of the trained VAE, and wherein the decoder generates a predicted expression profile using initial expression levels of the set of transcription factors;
adjusting one or more of the initial expression levels to obtain adjusted expression levels for the set of transcription factors;
using the decoder to generate an adjusted predicted expression profile from the adjusted expression levels, wherein the adjusted predicted expression profile corresponds to a current cell state; and
determining a difference metric between the current cell state and the target cell state by comparing the adjusted predicted expression profile to the target expression profile.

2. The method of claim 1, further comprising:
identifying a subset of the transcription factors that are adjustable for determining one or more candidate perturbation strategies to reach the target cell state.

3. The method of claim 2, further comprising:
determining whether to further adjust the expression levels for the subset of the transcription factors based on the difference metric.

4. The method of claim 2, further comprising:
performing a search by iteratively adjusting the subset of the transcription factors; and
based on the search, recommending a perturbation strategy from a list of candidate perturbation strategies, wherein the recommended perturbation strategy provides a best match of the predicted expression profile to the target expression profile corresponding to the target cell state.

5. The method of claim 4, further comprising:
implementing the perturbation strategy on a physical cell having the initial cell state.

6. The method of claim 1, wherein the VAE is trained using a VAE loss term that constrains the latent space of the VAE to match the initial expression levels.

7. The method of claim 1, wherein the set of transcription factors correspond to at least five target genes.

8. The method of claim 1, wherein the set of transcription factors comprises at least five transcription factors.

9. The method of claim 8, wherein the set of transcription factors comprises between five and 300 transcription factors.

10. The method of claim 1, wherein the initial cell state is a diseased state and the target cell state is an apoptotic state.

11. The method of claim 1, wherein the initial cell state is a diseased state and the target cell state is a healthy state.

12. The method of claim 10, wherein the diseased state is a cancerous cell state.

13. The method of claim 1, wherein the initial cell state corresponds to a first type of cells and the target cell state corresponds to a second type of cells, and wherein the first type and the second type are different.

14. The method of claim 13, wherein the first type of cells or the second type of cells are epithelial cells, immune cells, neural cells, muscle cells, stem cells, bone cells, blood cells, or fat cells.

15. The method of claim 1, wherein the decoder includes multiple fully connected layers, or a sparsely connected set of connections reflecting gene modules, pathways, or transcription factor regulons, of a neural network.

16. A method comprising performing, by a computer system:
loading an encoder of a variational autoencoder (VAE) into memory, wherein the encoder generates a latent space using an initial expression profile that comprises expression levels for a set of transcription factors;
loading a decoder of the VAE into the memory, wherein the decoder generates a predicted expression profile using the latent space; and
training the encoder and the decoder using a training set of expression profiles defining training cell states, wherein the training uses (1) a loss term including a difference between initial expression profiles and predicted expression profiles for the training set of expression profiles, and (2) a penalty term that constrains the latent space to match the expression levels for the set of transcription factors of the training set.

17. The method of claim 16, wherein the set of transcription factors correspond to at least five target genes.

18. The method of claim 16, wherein the set of transcription factors comprises at least five transcription factors.

19. The method of claim 18, wherein the set of transcription factors comprises between five and 300 transcription factors.

20. The method of claim 16, wherein parameters of VAE are updated in response to the loss term and the penalty term so as to further adjust the predicted expression profile.

21. The method of claim 16, wherein the decoder includes multiple fully connected layers of a neural network.

22. The method of claim 16, wherein the training cell states comprise an initial cell state and a target cell state.

23. The method of claim 22, wherein the initial cell state is a diseased state and the target cell state is an apoptotic state.

24. The method of claim 22, wherein the initial cell state is a diseased state and the target cell state is a healthy state.

25. The method of claim 23- or 24, wherein the diseased state is a cancerous cell state.

26. The method of claim 22, wherein the initial cell state corresponds to a first type of cells and the target cell state corresponds to a second type of cells, and wherein the first type and the second type are different.

27. The method of claim 26, wherein the first type of cells or the second type of cells are epithelial cells, immune cells, neural cells, muscle cells, stem cells, bone cells, blood cells, or fat cells.

28. A computer product comprising a non-transitory computer readable medium storing a plurality of instructions that, when executed, cause a computer system to perform the method of claim 1.

29. A system comprising:
the computer product of claim 28; and
one or more processors for executing instructions stored on the non-transitory computer readable medium.

30-32. (canceled)
